(19) United States
(12) Patent Application Publication
(10) Pub. No.: US 2026/0257037 A1
(43) Pub. Date: Sep. 3, 2026

(54) MULTI-FUNCTIONAL CATHETER SYSTEM WITH SELF-CENTERING ADAPTER FOR USABLE-LENGTH EXTENSION

(71) Applicant: CTI Vascular AG, Neuhausen (CH)

(72) Inventors: Marc GIANOTTI, Wiesendangen (CH); Dragana MARGETA, Stetten (CH); Michael JETTER, Thayngen (CH); Elisabeth SEEL, Klettgau-Erzingen (CH); Deborah LUETHI, Rüdlingen (CH); Kathrin MEIER, Winterthur (CH); Marco KELLER, Frauenfeld (CH); Andreas BODMER, Windisch (CH); Mejdina LOZIC, Schaffhausen (CH)

(73) Assignee: CTI Vascular AG, Neuhausen (CH)

(21) Appl. No.: 18/873,458

(22) PCT Filed: Jun. 10, 2022
(86) PCT No.: PCT/EP2022/065855

§ 371 Date: Dec. 10, 2024

(51) Int. Cl. A61M 25/00; A61M 25/01; A61M 25/09

(52) U.S. Cl. CPC A61M 25/0043; A61M 25/0108; A61M 25/09; A61M 2025/0004; A61M 2025/09133

(57) ABSTRACT

The current document is directed to a multi-functional catheter system (100) with self-centering adapter (130) for usable-length extension and methods that employ multi-functional catheter systems with self-centering adapters for usable-length extension to treat malformations, constrictions, obstructions, lesions, and blockages within patients' blood vessels. The multi-functional catheter system (100) comprises an integrated, slideable adapter (130) that self-centers onto a proximal end of the distal catheter shaft (120) of the multifunctional catheter system (100), and is configured for variable placement in an unprotected puncture site. Thereby, the self-centering adapter (130) enables the atraumatic insertion, maneuvering and removal of the multi-functional catheter system, in particular the proximal catheter extension shaft, without the need of an additional introducer or sheath. Further, the usable length of the multi-functional catheter system (100) with self-centering adapter (130), to which the current application is directed, can be extended over a set of lengths prior to and during medical procedures and is provided in a guidewire configuration that enables the simultaneous or consecutive use of guidewires without loss of access to a treatment site or requiring the exchange of one or more guidewires and catheters.

### TECHNICAL FIELD

The current document is directed to a multifunctional catheter system for the treatment of vascular pathologies and, in particular, to a multifunctional catheter system comprising an integrated, slideable adapter, that self-centers onto a distal catheter shaft of the multifunctional catheter system and in turn can be variably placed in an unprotected puncture site of a patient to be treated, wherein the multifunctional catheter system is configured for usable-length extension for treating a variety of different vascular conditions.

### BACKGROUND

Different types of catheter systems have been developed to treat a variety of different manifestations of vascular disease and other conditions within patients' veins and arteries that, when not treated, often lead to increasingly serious health conditions and complications, including ischemia, heart attacks, embolisms, and strokes. Contemporary diagnostic and therapeutic interventions for the treatment of vascular conditions are carried out using minimally invasive catheter devices that are administered percutaneously into a patient's vasculature. For enabling vascular access, a treatment provider performs a puncture at a variety of different blood-vessel access points, including the femoral, subclavian, radial, and brachial arteries. The treatment provider then inserts a guidewire through the puncture site into the blood vessel, and places an introducer or sheath in the wound canal, so that the catheter can be safely delivered into the blood vessel and advanced in or near the target region of the blood vessel to be treated.

Catheter systems are versatile and multi-functional, in that they can be used in combination with other specific medical devices, including for example guidewires, dilators, filters, stents and other catheter instruments, such as guiding-, angioplasty-, crossing-, atherectomy- or aspiration catheters, in addition to delivering diagnostic and therapeutic agents, such as embolization coils. Among the broad range of functionality exhibited in vascular interventions, catheters systems exemplarily serve to provide for substantial structural guidance and support as an external tubular shield, protect surrounding vasculature from trauma, and enable, among others, the maneuvering, centering and/or anchoring of co-administered devices, the targeted delivery of diagnostic and therapeutic agents within a region to be treated, as well as flushing and aspiration functionality.

However, currently available catheter systems have fixed usable lengths, referring to the length portions of the instrument shaft that are inserted into a patient. As a result, a treatment provider generally selects dimensionally compatible pairs of guide-wires and catheters of anatomically adequate length in order to access a particular treatment site from a particular access point. However, in many procedures, a vessel has to be treated at more than one site. In many cases, the treatment provider therefore needs to employ two or more catheters of two or more different lengths in order to reach the two or more treatment sites from the particular access point. Because catheter-based procedures involve prior insertion of the guidewire, insertion and removal of multiple, different-length catheters may result in a variety of problems, including a need to remove and reinsert different-length guide-wires, difficulties associated with maintaining sterile protocols across multiple sub-procedures, increased procedure times, and potential for additional complications arising from additional procedural steps, including prolonged radiation exposure and contrast agent dosing.

A significant advance in dealing with the above issues was gaining the ability to exchange medical devices over a single indwelling guide-wire without requiring displacement of the wire during the procedure and loss of access to the site. This “over the wire” (OTW) exchange technique requires an extra-long guide-wire so that control over the wire would be maintained at all times during the procedure. Operating the catheter over such a long wire is cumbersome and always associated with a risk of making the catheter or the wire unsterile.

Although the “long wire” or OTW technique still remains a commonly method of exchanging devices, other techniques were developed which allowed for a much shorter guide-wire and more physician control over the wire. In general, there are three further types of wire-catheter systems:
1. multi-exchange (MX)
2. rapid exchange (RX) and
3. fixed wire (FW).

OTW, MX and RX catheters require the use of a guide-wire that is separate from the catheter while a FW catheter has an integral guide-wire. An OTW catheter comprises a guide-wire lumen that extends the entire length of the catheter. The guide-wire is disposed entirely within the catheter guide-wire lumen except for distal and proximal portions of the guide-wire, which extend beyond the distal and proximal ends of the catheter, respectively. An MX catheter is arranged such that it has an over-the-wire configuration while the catheter is within the patient's body, but the wire exits the side of the catheter through a longitudinal slit configuration at a location outside the body.

OTW and MX catheters provide a full length guide-wire lumen, whereas RX catheters provide a short guide-wire lumen only at or near the distal end. However, traditional OTW, MX and traditional RX catheters do suffer some shortcomings as described above. RX catheters were developed in attempt to simplify the procedure for exchanging catheters over shorter guidewires. Catheters of this type are formed so that the guide-wire is located outside of the catheter except of a short guide-wire lumen that extends within only a comparatively short distal segment of the catheter. However, the use of shorter guide-wires reduces the effective reach to treatment sites within the vasculature, severely limiting their scope of applicability.

MX catheters include a longitudinal slit extending most of the length of the catheter, from which a wire may be removed laterally, or “peeled off”. At the distal end of the catheter is a short segment lacking the longitudinal slit, similar in nature to the short segment of the RX catheter. However, the required “peeling-off” procedure is tedious as it requires holding onto the proximal end of the wire, and careful removal is required, as great care must be taken to ensure that the peeling off procedure does not damage the wire. The catheter is likely damaged in the removal process and unfit for reinsertion.

While treatment providers benefit from the described guidewire exchange methods for remedying the applicational constraints of conventional fixed-length catheter devices, the limitation remains in that the catheters are typically offered in only one guidewire exchange configuration (e.g. RX or OTW), but not multiple at the same time. As instrument length and guidewire exchange configuration effectively limit the selection of interoperable instruments and guidewires that in turn determine available access lengths, the treatment provider therefore continues to be severely limited in the overall procedural versatility.

A recent advance in dealing with the above issues has been disclosed in the following prior art document. EP 3177356 (B1) is addressed to the technical problem of length-adjusting a balloon catheter. The balloon catheter comprises a catheter shaft wherein the length of its shaft can be varied over a length-adjustment range. In the preferred solution, the length adjustability is obtained by sliding a smaller diameter tube into a larger diameter tube while maintaining fluid impermeability across a portion of an inflation lumen contained within the smaller-diameter and larger-diameter tubes.

However, in the above example, the provided solution is limited to balloon catheters, and while the device can be operated in either RX or OTW mode, both guidewire exchange modes cannot be carried out simultaneously. Furthermore, the device requires the use of an additional introducer sheath, so that during a procedure the length-adjustment capability can be fully utilized. If such system were to be used without an introducer sheath, only the distal shaft portion of the catheter containing the larger-diameter tube would effectively be insertable through an access site without causing bleeding or trauma at the puncture site. In the absence of such introducer, once the smaller-diameter tube has been inserted, a dimensional gap would be formed between the shaft surface of the smaller-diameter tube and the surrounding tissue accessing the blood vessel. As a result, potential blood leakage at the puncture site would not only require additional attention by the treatment provider, but in general impair the efficacy of the procedure and potentially cause harm to the patient. More severe, upon removal of the device, the edge formed between the larger-diameter and smaller diameter tube could tear into unprotected tissue at the puncture site, leading to additional trauma to the patient. The above described example therefore requires an introducer sheath to fully utilize the length-adjustment capability, and in order to protect the puncture site of a patient. However, as introducers are typically provided with a tapered distal end to ease atraumatic insertion into a puncture site, the edge formed between the larger-diameter and smaller diameter tube may prevent removal of the larger-diameter tube through conventional introducers. Further, because introducers and/or sheaths are only available in fixed diameters and lengths, the combination and selection of available, interoperable catheter systems is even more restricted.

In view of the above considerations it is still desirable to provide a catheter system that facilitates the selection and adjustment of the usable length of the catheter during the medical procedure without having the limitations or drawbacks of the known OTW, MX, RX and FW systems. Furthermore, it would be desirable to provide a catheter system that combines the benefits of the known systems but nevertheless allows an improved handling and easy adjustment of its usable length. In addition, it would be desirable to provide a catheter system that facilitates the usable length to be selected and adjusted during the medical procedure, without the requirement of removing and exchanging the catheter and/or the wire-guide. In particular, it would be highly desirable to provide a catheter system that allows the usable length of the system to be selected and changed during the medical procedure without requiring an additional introducer or sheath, and that facilitates a variably adaptable protection of a puncture site by allowing a treatment provider to adapt the system to one or more diameters and lengths present at a puncture site. Finally, it would be desirable to provide a catheter system that increases a range of compatible diameters between the catheter system and adjunct medical devices, and that permits the introduction and removal of the catheter system at short, medium and long access lengths (distances from a puncture site to a treatment site).

However, none of the known catheter systems comprise the certain combination of features of the catheter system of the present invention and, therefore, none of the known systems solve the above described problems.

The present inventors now found that the above problems can be solved by a multi-functional catheter system configured for usable-length extension comprising an integrated, slideable adapter that self-centers onto a distal catheter shaft of the multifunctional catheter system. The integrated, slideable adapter can in turn be variably placed in an unprotected puncture site of a patient to be treated, enabling a treatment provider to adapt the system to one or more diameters and lengths present at a puncture site. Thereby, the self-centering adapter facilitates the atraumatic insertion, maneuvering and removal of the multi-functional catheter system, and in particular, a proximal shaft component, without the need of an additional introducer or sheath, and increases a diameter compatibility range between the catheter system and adjunct medical devices. The multi-functional catheter system of the current disclosure enables the usable length to be selected and adjusted during the medical procedure for treating a variety of different vascular conditions at a broad range of access lengths, and further is provided in one or more guidewire configurations that enable the simultaneous or consecutive use of guidewires without loss of access to a treatment site or requiring the exchange of one or more guidewires and catheters.

### SUMMARY

The current document is directed to a multi-functional catheter system with self-centering adapter for usable-length extension and methods that employ multi-functional catheter systems with self-centering adapters for usable-length extension to treat malformations, constrictions, obstructions, lesions, and blockages within patients' blood vessels. The multi-functional catheter system comprises an integrated, slideable adapter that self-centers onto a distal catheter shaft of the multifunctional catheter system, and is configured for variable placement in an unprotected puncture site. Thereby, a treatment provider can variably adapt the system to one or more diameters and lengths present at a puncture site, and consecutively, when the multi-functional catheter is used without an additional introducer or sheath, enable the atraumatic insertion, maneuvering and removal of the multi-functional catheter system, and in particular, a proximal shaft component. Further, the self-centering adapter extends a diameter compatibility range of the catheter system and adjunct medical devices, and permits the variable introduction or removal of the catheter system at short, medium and long access lengths.

The usable length of the multi-functional catheter system with self-centering adapter, to which the current application is directed, can be adjusted over a set of lengths prior to and during medical procedures and is provided in one or more guidewire configurations that enable the simultaneous or consecutive use of guidewires without loss of access to a treatment site or requiring the exchange of one or more guidewires and catheters.

In additional implementations, usable length-adjustment of the multi-functional catheter system is accompanied by relative-position indications, to the medical provider, of the extent of a usable length selection. The indications may include one or more of visual markings, haptic feedback, radio-opaque markings, and/or other types of indications. In these implementations, the usable length-adjustment mechanism of the multi-functional catheter system is mechanically lockable following usable length selection.

### BRIEF DESCRIPTION OF THE DRAWINGS

FIG. 1 illustrates a perspective view of a first implementation of a multi-functional catheter system with self-centering adapter, in an assembled state.

FIG. 2A-B illustrate a cross-sectional view of contacting surfaces between a self-centering adapter and a distal catheter shaft of the multi-functional catheter system in a separated and in a contacting state.

FIG. 3 illustrates a cross-sectional view of contacting surfaces between a self-centering adapter and a hub of the multi-functional catheter system in a contacting state.

FIG. 4 illustrates a cross-sectional view of a first implementation of a self-centering adapter of the multi-functional catheter system.

FIG. 5 illustrates a cross-sectional view of a second implementation of a self-centering adapter of the multi-functional catheter system.

FIG. 6 illustrates a cross-sectional view of a third implementation of a self-centering adapter of the multi-functional catheter system.

FIG. 7 illustrates a cross-sectional view of a fourth implementation of a self-centering adapter of the multi-functional catheter system.

FIG. 8 illustrates a cross-sectional view of a fifth implementation of a self-centering adapter of the multi-functional catheter system.

FIG. 9 illustrates a cross-sectional view of a sixth implementation of a self-centering adapter of the multi-functional catheter system.

FIG. 10 illustrates a cross-sectional view of a seventh implementation of a self-centering adapter of the multi-functional catheter system.

FIG. 11 illustrates a cross-sectional view of a fluid-impermeable slideable seal that facilitates sliding of a proximal catheter extension shaft within a distal catheter shaft of the multi-functional catheter system.

FIG. 12 illustrates a cross-sectional view of a locking and position-maintaining feature between a proximal catheter extension shaft and a distal catheter shaft of the multi-functional catheter system.

FIG. 13A-C illustrate cross-sectional views of several implementations of torqueing features of proximal and distal shafts of the multi-functional catheter system.

FIG. 14 illustrates a cross-sectional view of a single-lumen catheter tip of the multi-functional catheter system, as a first implementation.

FIG. 15 illustrates a cross-sectional view of a single-lumen catheter tip of the multi-functional catheter system, as a second implementation.

FIG. 16 illustrates a cross-sectional view of a single-lumen catheter tip of the multi-functional catheter system, as a third implementation.

FIG. 17 illustrates a cross-sectional view of a dual-lumen catheter tip of the multi-functional catheter system, as a fourth implementation.

FIG. 18 illustrates a cross-sectional view of a dual-lumen catheter tip of the multi-functional catheter system, as a fifth implementation.

FIG. 19 illustrates shaping of a dual-lumen catheter tip of the multi-functional catheter system, as a sixth implementation.

FIG. 20A-D illustrate cross-lateral views representing four consecutive configurational stages A-D for operating the self-centering adapter and adjusting the usable length of the multi-functional catheter system, as several embodiments.

FIG. 21A-C illustrate cross-lateral views representing the various effects of placing a multi-functional catheter system into a puncture site of a patient in the absence or presence of the self-centering adapter.

FIG. 22A-E illustrate a series of steps carried out by a treatment provider, using a multi-functional catheter system configured for usable-length extension in combination with an integrated, self-centering adapter to access multiple treatment sites within a patient's vasculature.

### DETAILED DESCRIPTION

The current document is directed to a multi-functional catheter system with a self-centering adapter for usable-length extension and methods that employ multi-functional catheter systems with self-centering adapters for usable-length extension to treat malformations, constrictions, obstructions, lesions, and blockages within patients' blood vessels. The multi-functional catheter system comprises an integrated, slideable adapter that self-centers onto a proximal end of the distal catheter shaft of the multifunctional catheter system, and is configured for variable placement in an unprotected puncture site, which enables a treatment provider to adapt the system to one or more diameters and lengths present at the puncture site. Thereby, the self-centering adapter enables the atraumatic insertion, maneuvering and removal of the multi-functional catheter system, and in particular, a proximal catheter extension shaft, without the need of an additional introducer or sheath. Because the self-centering adapter is directly seated on a proximal catheter extension shaft of the multi-functional catheter system, in turn the profile of the multi-functional catheter of the current disclosure can be significantly smaller than compared to conventional catheter systems that are deployed in combination with a conventional introducer. The multi-functional catheter system therefore permits the use of smaller-sized catheter profiles that in turn expand an accessible diameter range or compatibility between the multi-functional catheter system and any adjunct medical devices that can be deployed in combination with the multi-functional catheter system. Therefore, the integrated slideable adapter extends a diameter compatibility range of the catheter system and adjunct medical devices, and permits the variable introduction or removal of the catheter system at short, medium and long access lengths. The multi-functional catheter system in turn facilitates the selection and adjustment of the usable length that is applicable to render treatments of single target sites within blood vessels as well as procedures that involve treatment of multiple target sites. Because the usable length of a multi-functional catheter configured for usable-length extension can be selected and adjusted prior to and during a medical procedure, after the catheter has been initially inserted into a patient's blood vessel, the multi-functional catheter system configured for usable-length extension provides for adjustment of initial non-optimal placements and/or guidewire to catheter length mismatches, changes to which might otherwise involve removal of an initially inserted first catheter and/or guide-wire and reinsertion of a second catheter and/or guide-wire with a different length. Further, the system is provided in one or more guidewire exchange configurations that enable the simultaneous or consecutive use of guidewires without loss of access to a treatment site or requiring the exchange of one or more guidewires and catheters. Usable-length-adjustability, combined with sheath-less procedures, and simultaneous or consecutive use of the catheter system in RX and/or OTW mode enable the catheter system to significantly simplify treatments of multiple target sites within a blood vessel, since the usable length of the multi-functional catheter can be changed, prior to and during a procedure, following treatment of a first target site, in order to reposition the catheter to treat a second or consecutive target site. Multi-functional catheters configured for usable length extension may also reduce needed equipment inventories, since fewer different sized multi-functional catheters are needed to span the potential range of usable lengths needed for accessing variably positioned treatment sites encountered in human anatomy. Selecting the access site for a procedure is crucial for a treatment provider in that choosing a specific puncture site limits the overall procedural versatility and available access length in the anatomy to be treated. For example, a radial approach permits faster ambulation of a patient, and at the same time, provides the physician a much greater versatility for the procedure due to a shortened access length and ease of access as compared to a femoral approach. Because multi-functional catheter systems configured for usable-length extension provide for an enhanced access range in the vascular anatomy, the treatment provider can select from a much broader spectrum of available access sites. Further, treatment providers may opt to select guide-wires that are shorter in length than the total usable length of the catheter, thereby providing significant handling improvement while still having the option to utilize the full total usable length of the instrument when needed. In the following discussion, examples of multi-functional catheter systems with self-centering adapters configured for usable-length extension are illustrated and described. The usable-length-adjustability discussed with reference to the various implementations of multi-functional catheter systems can be applied to, and incorporated within, other types of catheters used for diagnostic and therapeutic procedures. While the description set forth below describes the multi-functional catheter system and its components in connection with its application to blood vessels, or vascular applications, it is understood that the multi-functional catheter system may in principle also be used in other body cavities, for example in non-vascular applications, if so desired.

### Catheter System

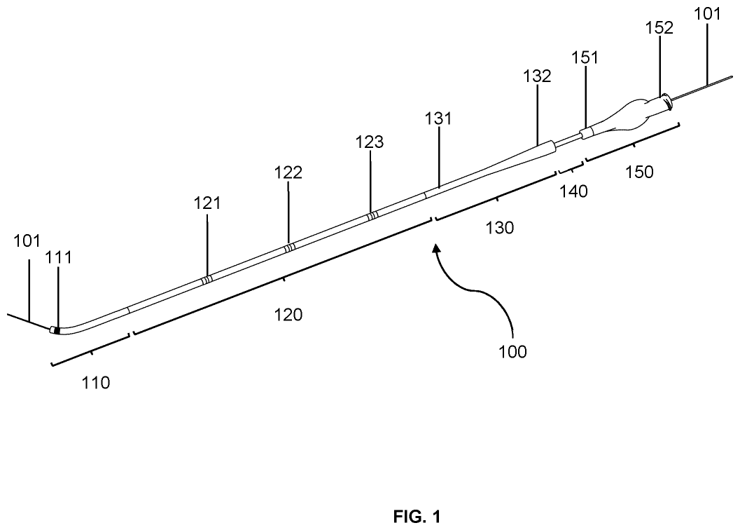

The various components and features of the multi-functional catheter system with self-centering adapter for usable length extension are next described with reference to FIG. 1. FIG. 1 illustrates a perspective view of a first implementation of a multi-functional catheter system with self-centering adapter, in an assembled state. In FIG. 1, the multi-functional catheter system 100 comprises, from left to right, a catheter tip 110, a distal catheter shaft 120, a self-centering adapter 130, a proximal catheter extension shaft 140, and a hub 150.

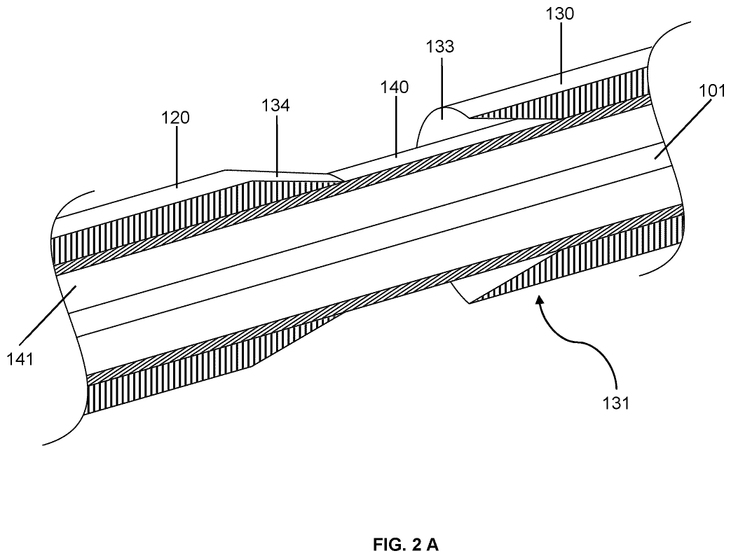

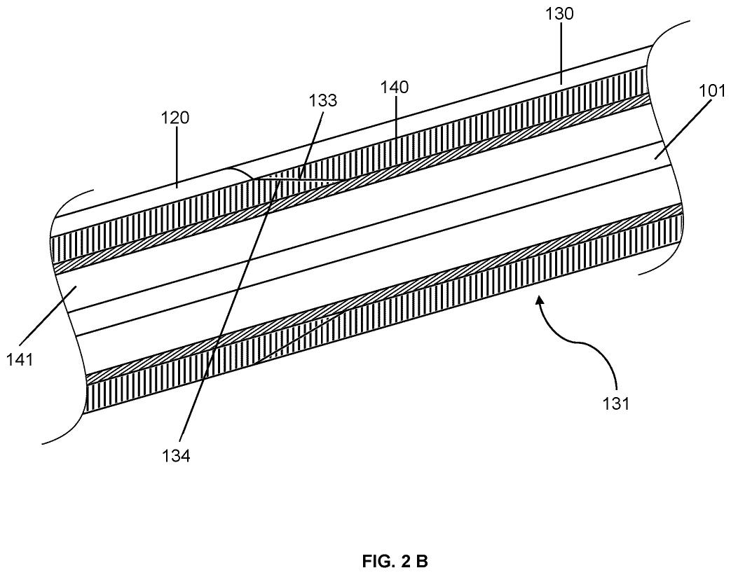

The adapter 130 is slideably received on the proximal catheter extension shaft 140 and configured to couple to a distal end 151 of the hub 150 in a first or resting position, such that the proximal catheter extension shaft 140 is kink protected. The adapter is further configured to couple to a proximal end of the distal catheter shaft in a second or docking position, such that a resulting transition between a diameter of the adapter and a diameter of the distal catheter shaft is edge- and seamless. In the second position, the adapter 130 is configured for variable placement in an unprotected puncture site of a patient to be treated. Generally, the adapter 130 covers a range of outer diameters and lengths that allow a treatment provider to variably adapt to, and thereby protect, one or more diameters and lengths present at a puncture site. In turn, variable placement of the adapter into the puncture site of a patient enables hemostatic sealing, among others. When the adapter 130 is coupled to the proximal end of the distal catheter shaft 120, and placed in the puncture site, the proximal catheter extension shaft of the multi-functional catheter system is movably centered in the puncture site without having direct contact thereto. Thereby, the puncture site becomes protected. Further, when the proximal catheter extension shaft is retracted proximally by a treatment provider, the self-centering adapter is able to ‘funnel’ onto the proximal end of the distal catheter shaft without requiring direct user interaction, thereby shielding the puncture site from bleeding or trauma when a diameter gap exists between the proximal and distal catheter shaft. Hence, as a specific technical feature, the adapter enables the atraumatic insertion, maneuvering and removal of the proximal catheter extension shaft of the usable-length-adjustable catheter system 100 without the need of an additional introducer or sheath. For further orientation, additional details of the adapter will be discussed in reference to FIGS. 2-10.

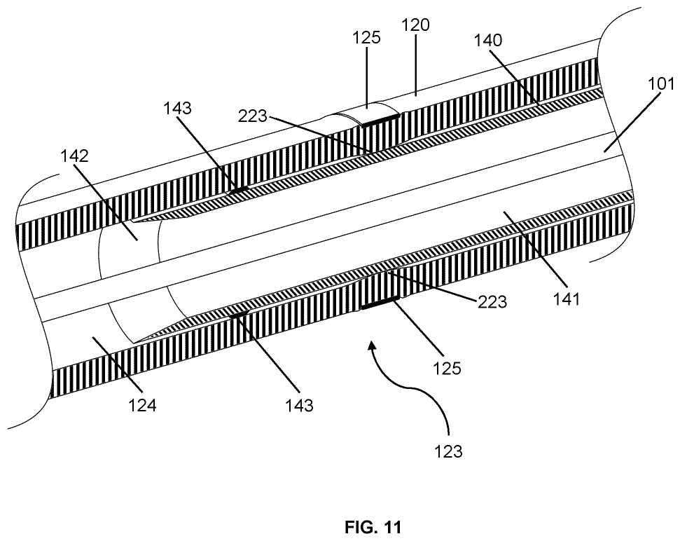

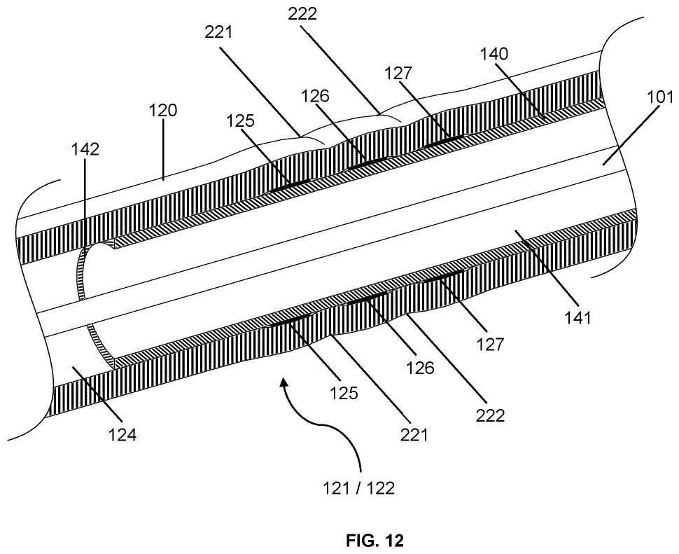

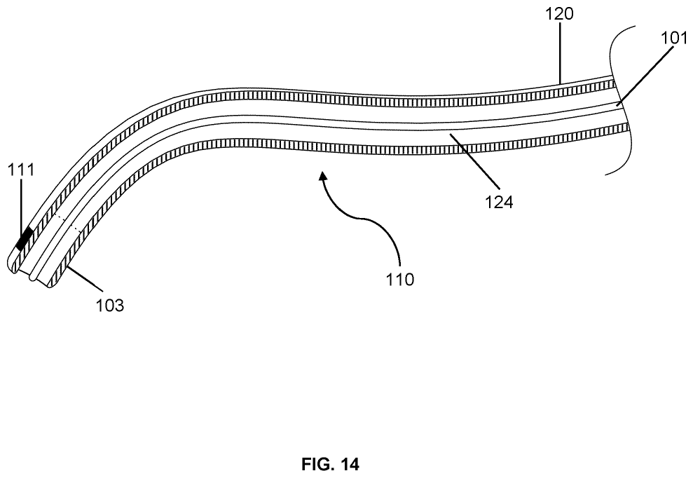

The proximal catheter extension shaft 140 is slideably received within a portion of the distal catheter shaft 120 and configured to extend the usable length of the multi-functional catheter system from a contracted to one or more extended positions based on a treatment provider's usable-length selection. Each catheter shaft 120 and 140 of the multi-functional catheter system further includes one or more guidewire lumen, that extend from an opening or guidewire port at the distal end of the catheter tip 110 to an opening or guidewire port at the hub 152 of the catheter. The opening at the hub 152 and the opening at the distal end of the catheter tip 110 therefore constitute two or more guidewire ports, respectively. Additional details of the guidewire lumen 141 contained in the proximal catheter extension shaft 140 and the guidewire lumen 124 contained in the distal catheter shaft 120 will be discussed in reference to FIGS. 11-12, and specific guidewire lumen and catheter tip configurations will be discussed in reference to FIGS. 14-19.

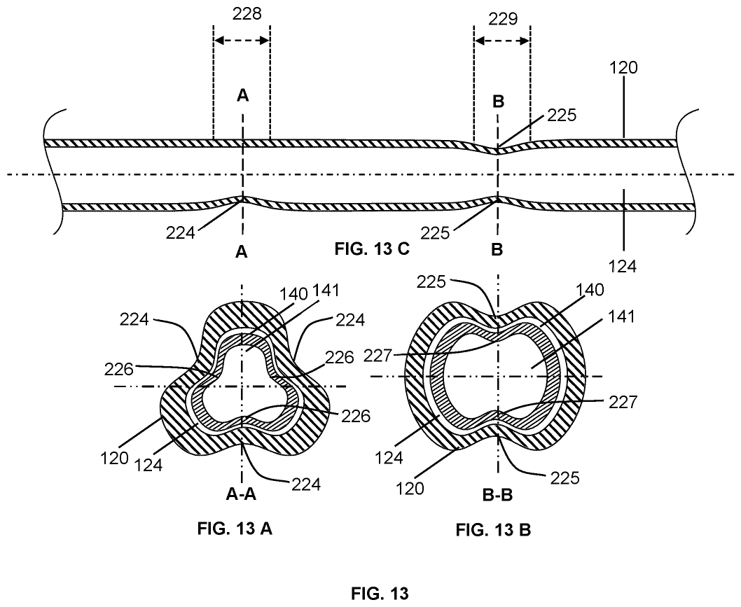

For usable length selection and adjustment, both proximal catheter extension shaft 140 and distal catheter shaft 120 include two or more sealing and position-maintaining features 121, 122, and 123 that maintain one or more positions of the proximal catheter extension shaft 140 inside the distal catheter shaft 120 while maintaining fluid impermeability across one or more guidewire lumen. Additional details of the sealing and position-maintaining features will be discussed in reference to FIGS. 11-12. Further, the multi-functional catheter system 100 includes orientation maintaining features that allow the transmission of torque or rotational forces exerted by a treatment provider from a proximal catheter extension shaft 140 to a distal catheter shaft 120, and thereby to a catheter tip 110, so as to enable enhanced steerability of the system. For further orientation, details of the torqueing features will be discussed in reference to FIG. 13.

Concerning the general construction aspects of the multi-functional catheter system, the catheter components can be manufactured from biocompatible, polymeric, metallic and ceramic materials. For example, the catheter components may be manufactured from aliphatic, semi-aromatic and aromatic polyamides (PA); polyether ether ketones (PEEK); polyimides; linear and nonlinear, branched or non-branched, low molecular weight, medium molecular weight, or high molecular weight; low density, medium density, or high density polyolefins, including polyethylene (PE) and polypropylene (PP), silicones, thermoplastic elastomers, such as polyurethanes (TPEs) and fluoroelastomers, for example FEP or PTFE, polycarbonates (PC), polyethylene terephthalate (PET) and combinations, including blends and copolymers of any of these materials.

Further, the catheter components can also be fabricated as a single layer, dual-layer, or in multi-layer configuration. In the instance of dual-layer or multi-layer configurations, certain catheter elements, including for example the shaft, may utilize the same material for each layer or may utilize different materials for each layer. The multiple layers may be glued, melted or fused together with or without an adhesive, or by employing a co-extrusion or welding process. Alternatively, the multiple layers are not required to be attached, glued or welded together; instead, the multiple layers may be allowed to move independently. Additionally, the elastic modulus, durometer or hardness of the materials selected for each layer or component of the catheter system can be varied to beneficially alter the performance aspects of the individual catheter components.

Also, the chemical functionality and/or physical polarity of the catheter materials can be changed to enhance interfacial adhesion between the differing layers and/or to provide surfaces and/or inner lumen with an increased lubriciousness or changed surface energy when in contact with guide wires, therapeutic and diagnostic liquids, or functional coatings, for example. These chemical and physical treatments or alternations may include for instance chemical additives that can introduce another chemical functionality to the interfacial surface, when added to an exemplary base polymer formulation intended to form one or more layers of the catheter component, for example, including functional groups such as carboxy- and/or amino groups, which can effectively enhance the underlying polarity of the layer and the substrate, thus facilitating enhanced adhesion and mechanical fixation strength in between one or more layered structures of catheter components.

Other surface modifications, such as coatings and/or plasma techniques can be employed for further changing the chemical and/or the mechanical properties of the materials, layers or components of the catheter system, wherein the modification of the catheter materials may affect the polarity, surface energy and/or friction coefficient of layers and/or surfaces of the catheter components. Other suitable techniques may incorporate additives, adhesives and/or filling agents, which can introduce other beneficial properties to the catheter materials. For example, the components of the catheter system may incorporate radiopaque elements embedded within polymeric materials to selectively increase fluoroscopic visibility at desired locations. Alternatively, or supplementary, the components of the catheter system may incorporate dyes or pigments at select locations to provide visible color-indications to a treatment provider. Additionally, the shaft may incorporate fluoropolymer-based filler particles/fibers to permanently decrease the frictional coefficient as compared to an untreated base-polymer formulation or activatable, single-use coatings. Furthermore, the shaft can be provided reinforced and may contain metal or polymer-based strands, fibers, wires, braids, meshes and/or fabrics embedded as layers, sections or regions into the base-shaft material.

In summary, the multi-functional catheter system 100 comprises:
a hub 150;
a guidewire lumen 124, 141;
a proximal catheter extension shaft 140;
a distal catheter shaft 120;
a catheter tip 110;
two or more guidewire ports 152, and
a self-centering adapter 130;
wherein the adapter 130 is slideably received on the proximal catheter extension shaft 140 and configured to couple to a distal end 151 of the hub 150 in a first or resting position, such that the proximal catheter extension shaft 140 is kink protected; and
wherein the adapter 130 is further configured to couple to a proximal end 134 of the distal catheter shaft 120 in a second or docking position, such that a resulting transition between a diameter of the adapter and a diameter of the distal catheter shaft is edge-and seamless; and
wherein in the second position, the adapter 130 is configured for variable placement in an unprotected puncture site 402 of a patient to be treated, such that the proximal catheter extension shaft of the multi-functional catheter system is movably centered in the then protected puncture site without direct contact thereto;
characterized in that the adapter 130 thereby enables the atraumatic insertion, maneuvering and removal of the proximal catheter extension shaft of the multi-functional catheter system without the need of an additional introducer. Thereby, the multi-functional catheter system 100 comprising a self-centering adapter 130 does not require the presence of an additional introducer to enable the atraumatic insertion, maneuvering and removal of the multi-functional catheter system, and in particular a proximal catheter extension shaft 140.

### Adapter Configuration

FIG. 2A-B illustrate a cross-sectional view of contacting surfaces between a self-centering adapter and a distal catheter shaft of the multi-functional catheter system in a separated and in a contacting state. In FIG. 2A, the adapter 130 is shown slideably received on the proximal catheter extension shaft 140 that, in turn is slideably received within a portion of the distal catheter shaft 120. A guidewire 101 extends across a guidewire lumen 141 of the proximal catheter extension shaft 140. The distal end 131 of the adapter 130, which is shown in a first position separate from the proximal end of the distal catheter shaft 120, comprises a contact surface 133 at the distal end 131 of the adapter 130 that tapers inwards from an outer diameter of the adapter at a distal end 131 to an outer diameter of the proximal catheter extension shaft 140. Facing the distal end of the adapter 131 is a contact surface 134 at the proximal end of the distal catheter shaft 120 that tapers from an outer diameter of the distal catheter shaft 120 to an outer diameter of the proximal catheter extension shaft 140. The adapter 130 is formed as a tubular member that comprises a distal end or tip 131, a mid-section and a proximal end 132.

In FIG. 2B, the contacting surface 133 at the distal end 131 of the adapter 130 and the contacting surface 134 at the proximal end of the distal catheter shaft 120 are shown in a contacting state. In FIG. 2B, the adapter cross-section readily indicates that an outer diameter of the adapter 130 at a distal end 131 is equivalent to an outer diameter of the distal catheter shaft 120 and an inner diameter of the adapter is equivalent to an outer diameter of the proximal catheter extension shaft 140. Further, the contact surface 134 at the proximal end of the distal catheter shaft 120 tapers from an outer diameter of the distal catheter shaft 120 to an outer diameter of the proximal catheter extension shaft 140, and the contact surface 133 at the distal end 131 of the adapter 130 tapers inwards from an outer diameter of the adapter at a distal end 131 to an outer diameter of the proximal catheter extension shaft 140. In turn, because the tapering angles and respective inner and outer dimensions of the contacting surfaces 133 and 134 are congruent with each other, the adapter 130 is configured to couple to the proximal end 134 of the distal catheter shaft 120. As a result of this arrangement, in this second or docking position of the adapter, the transition between the outer diameter of the adapter and the outer diameter of the distal catheter shaft is both edge-and seamless. Thereby, when the adapter is positioned in a puncture site of a patient to be treated, an outer diameter disparity between a proximal catheter extension shaft and a puncture site does not cause bleeding or trauma, as compared to conventional catheter systems.

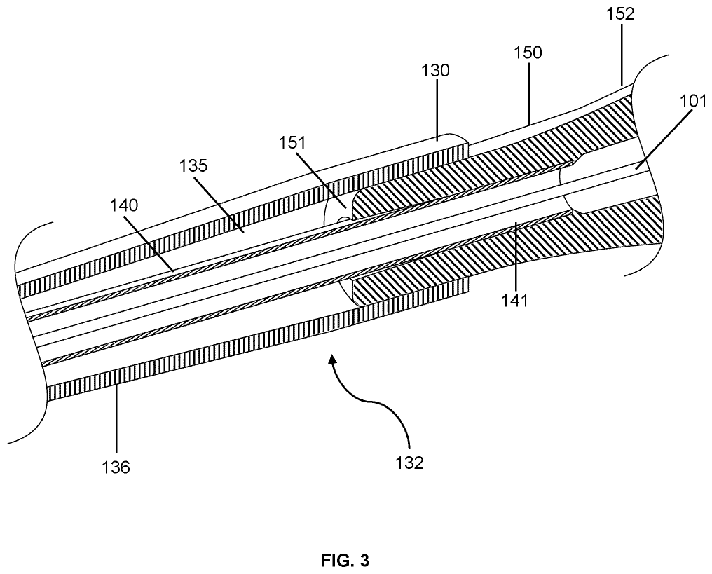

FIG. 3 illustrates a cross-sectional view of contacting surfaces between a self-centering adapter and a hub of the multi-functional catheter system in a contacting state. In FIG. 3 the adapter 130 is shown slideably received on the proximal catheter extension shaft 140 that in turn is fixedly attached to the hub 150. A guidewire 101 extends across a guidewire lumen 141 of the proximal catheter extension shaft 140 through an opening or guidewire port at the proximal end of the hub 152. The proximal end 132 of the adapter 130 is shown in the first or resting position, wherein an inner surface of the proximal end 132 of the adapter 130 is slid over or contacting an outer surface of the distal end 151 of the hub 150. In this first position, the distal end 131 of the adapter 130 is also separate from the proximal end of the distal catheter shaft 120 as described in reference to FIG. 2A. In FIG. 3, the adapter cross-section readily indicates that an outer diameter of the adapter 130 at a proximal end 132 exceeds an outer diameter of a distal end 151 of the hub 150. Further, an inner diameter of the adapter 130 at the proximal end 132 is equivalent or larger than an outer diameter of the distal end 151 of the hub 150, and an outer diameter of the mid-section 136 tapers between the outer diameters of the adapter at the distal and proximal end. In turn, because the respective inner and outer dimensions of the contacting surfaces of the proximal end 132 of the adapter 130 and the distal end 151 of the hub 150 allow the adapter to be slideably received over the distal end 151 of the hub 150, the adapter is thereby configured to couple to a distal end 151 of the hub 150 in a first or resting position. As a result of this arrangement, in the first or resting position of the adapter, the proximal catheter extension shaft 140 is kink protected.

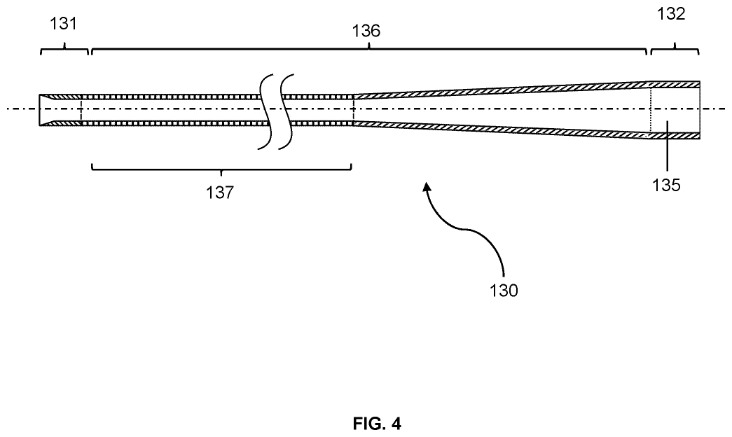

FIG. 4 illustrates a cross-sectional view of a first implementation of a self-centering adapter of the multi-functional catheter system. In FIG. 4, the adapter 130 is formed as a hollow, tubular member 135 that comprises a distal end 131, a mid-section 136 and a proximal end 132. In the first implementation, the proximal end 132 of the adapter 130 is formed as a cylindrical portion that is capable of sliding over a distal end 151 of the hub 150. The midsection 136 extends from the distal end 132 of the adapter to the proximal end 131 of the adapter, and an outer diameter of the mid-section tapers between the outer diameters of the adapter at the distal and proximal end. Further, a transition 137 between the distal end and the mid-section of the adapter 130 is cylindrical.

In general, the taper and length of the mid-section 136 can be dimensioned so as to cover a range of diameters and lengths that allow the diameter and length of the adapter to variably adapt to one or more diameters and lengths present at a puncture site. Thereby, through variable placement or insertion of an adapter by a treatment provider into the puncture site of a patient, the taper or range of outer diameters of the adapter variably conforms to a range of diameters existing in the puncture site so as to enable hemostatic sealing. Additionally, forces exerted by insertion of the adapter onto the puncture site may vary as to the degree of insertion length selected by the treatment provider, so that the resulting forces have a stabilizing and/or position-maintaining effect on the adapter, as well as a sealing and/or a dilating effect on the puncture site. Further, the range of outer diameters covered by the taper of the mid-section 136 of the adapter can additionally be provided color-indicated on a surface of the adapter to visually aid a treatment provider in determining compatibility between an outer or inner diameter of an adapter and an outer or inner diameter of a puncture site, wound canal, blood vessel or an adjunct medical device. For example, a color-indication can be color-coded to correspond to French size units that are used to indicate sizes in medical procedures. Concerning inner dimensions of the adapter, the inner diameter is dimensioned with respect to the outer diameter of the proximal catheter extension shaft such that a diameter gap between them is sufficiently large so as to allow a substantially frictionless movement of the shaft inside the adapter and prevent air entrapment, and yet at the same time, sufficiently small to ensure minimal risk of leaking physiological, diagnostic or therapeutic fluids through said gap, when the adapter has been inserted into the puncture site of a patient to be treated.

In the first implementation, the proximal end 132 and the tapering portion of the mid-section 136 are formed from the same or first material, and shown seamlessly affixed to a cylindrical portion 137 of the mid-section 136 that is formed from a different or second material. The cylindrical portion 137 further extends into the distal end or tip 131 of the adapter that in turn is formed of yet another or third material.

In the various implementations, the adapter 130 is generally formed from thermoplastic elastomers. However, concerning the individual components of the adapter, the adapter is not limited as to the exact disposition of materials, for example, the distal end 131, mid-section 136, 137 and proximal end 132 of the adapter 130 can be formed from the same or different thermoplastic elastomers. Suitable thermoplastic elastomers of the adapter 130 include at least one or more of acrylonitrile butadiene styrene, polytetrafluoroethylene, fluorinated ethylene propylene, polyvinyl chloride, polyamide, polyurethane, polyethylene, low density polypropylene, high density polypropylene, polyethylene imine, and silicone.

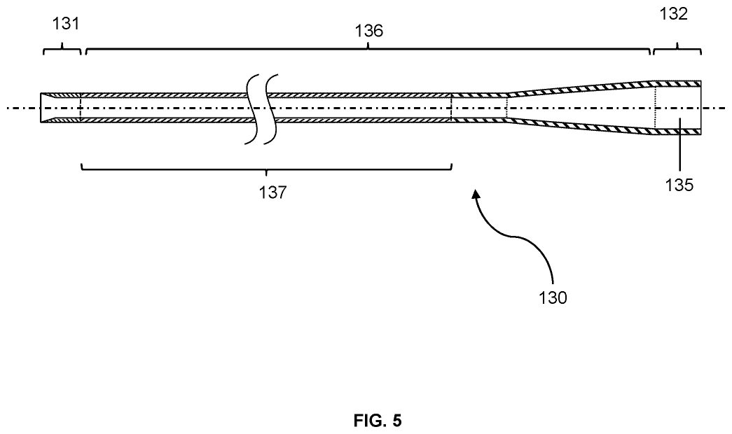

FIG. 5 illustrates a cross-sectional view of a second implementation of a self-centering adapter of the multi-functional catheter system. In FIG. 5, the adapter 130 is formed as a hollow, tubular member 135 that comprises a distal end 131, a mid-section 136 and a proximal end 132. In the second implementation, the proximal end 132 of the adapter 130 is formed as a cylindrical portion that is capable of sliding over a distal end 151 of the hub 150. The midsection 136 extends from the distal end 131 of the adapter to the proximal end 132 of the adapter, and an outer diameter of the mid-section tapers between the outer diameters of the adapter at the distal and proximal end. In comparison to the first implementation, in the second implementation the taper of the mid-section 137 transitions into a first cylindrical portion that is fixedly attached to a second cylindrical portion that is different in material from the first cylindrical portion.

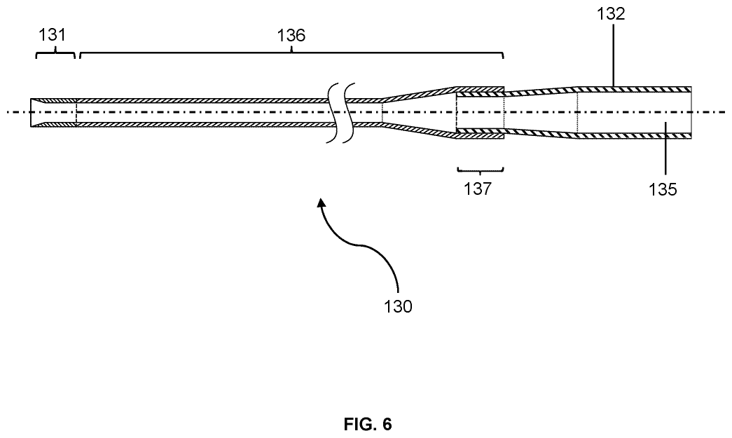

FIG. 6 illustrates a cross-sectional view of a third implementation of a self-centering adapter of the multi-functional catheter system. In FIG. 6, the adapter 130 is formed as a hollow, tubular member 135 that comprises a distal end 131, a mid-section 136 and a proximal end 132. In comparison to the previous implementations, in the third implementation the proximal end 132 of the adapter tapers into a first cylindrical portion 137 that in turn tapers into a second cylindrical portion of the mid-section 136, and is shown in a coaxial arrangement wherein an outer surface of the first cylindrical portion 137 is adhered onto an inner surface of the midsection 136. In FIG. 6, the transition or overlapping region 137 between the proximal end 132 and the mid-section 136 of the adapter 130 is formed from a malleable material and provided in a first shape that is elastically or plastically deformable to a second shape by an externally applied bending force thereby enabling shapeability of the adapter.

Accordingly, in the various implementations, the adapter can be formed from a combination of malleable and elastic elastomers that allow a section of the adapter to be temporarily or permanently be deformed to a suitable shape that aids a treatment provider in stabilizing and/or fixing a position and/or orientation of the adapter with respect to a blood vessel, wound canal, puncture site, and/or skin area of a patient to be treated, as well as with respect to co-administered diagnostic or therapeutic agents and/or adjunct medical devices.

Further, an elastic modulus or hardness of the distal end 131 of the adapter 130 can be lower than an elastic modulus or hardness of the mid-section 136 or transition 137 of the adapter; and an elastic modulus or hardness of the proximal end 132 of the adapter can exceed an elastic modulus or hardness of the mid-section 136 or transition 137 of the adapter, so as to provide variable degrees of flexibility and stiffness or soft-and hardness along the length of the adapter, which can aid a treatment provider in atraumatically inserting as well as positioning or orienting the adapter within a puncture site of a patient to be treated.

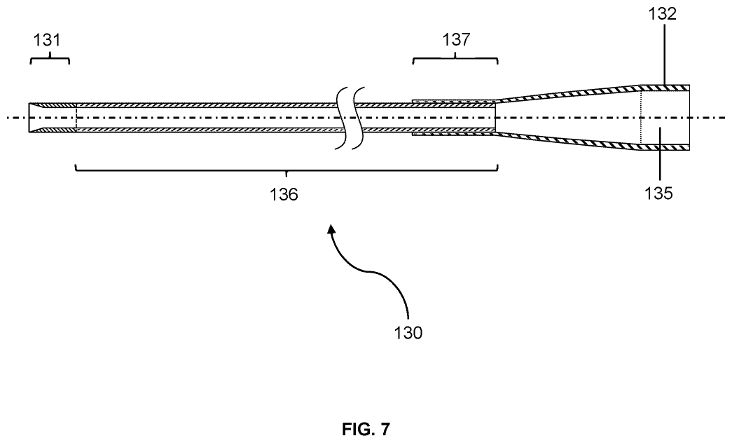

FIG. 7 illustrates a cross-sectional view of a fourth implementation of a self-centering adapter of the multi-functional catheter system. In FIG. 7, the adapter 130 is formed as a hollow, tubular member 135 that comprises a distal end 131, a mid-section 136 and a proximal end 132. In comparison to the previously shown implementations, in the fourth implementation the proximal end 132 of the adapter tapers into a first cylindrical portion 137 that in turn tapers into a second cylindrical portion of the mid-section 136, and is shown in a coaxial arrangement wherein an inner surface of the first cylindrical portion 137 is adhered onto an outer surface of the midsection 136. When the individual components of the adapter are engaged in a coaxial arrangement, such as provided in the above example, the components may be fixedly or slideably adhered to each other. When the components are not fixedly adhered to each other, the coaxially arranged components of the adapter may be slid into each other, or in other words, comprise length-adjustment features, so as to variably adapt the length of the adapter to one or more lengths present at the puncture site.

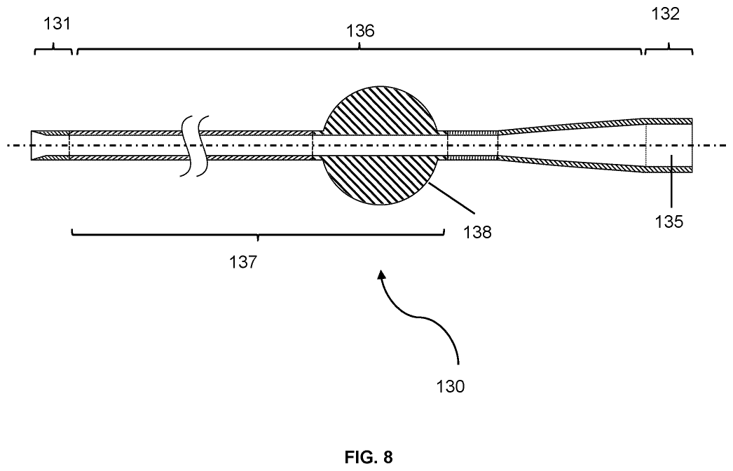

FIG. 8 illustrates a cross-sectional view of a fifth implementation of a self-centering adapter of the multi-functional catheter system. In its base construction, the fifth implementation of the adapter 130 is similar to the first implementation. However, the fifth implementation differs from the first implementation in that the adapter further comprises a spherical protrusion 138 positioned below and/or around a proximal end 132 of the adapter that during insertion of the adapter into a puncture site exerts a sealing force on an opening of the puncture site, thereby enabling hemostatic sealing. The spherical protrusion 138 is shown fixedly attached to the outer surface of the adapter and formed of a different material as the distal end 132. In FIG. 8, the outer diameter of the protrusion is shown larger than an outer diameter of the proximal end 132 of the adapter 130. However, depending on the typical diameter of a puncture site, and for ease of handling, the diameter of the protrusion can also be of equal or smaller outer diameter than the outer diameter of the proximal end 132 of the adapter 130. Further, the proximal end 132, mid-section 136 and cylindrical portion or transition 137 and distal end 131 of the adapter can be formed of the same or different materials that in turn exhibit the same or differing degrees of elastic modulus, durometer or hardness. In additional implementations, the spherical protrusion is provided with color-indications that indicate an inner or outer diameter or generally, a size aspect to a treatment provider. For example, the color can be indicative of a diameter or size, of an adapter, a spherical protrusion or other feature of an adapter, a puncture site, or refer to specific diameter compatibility, when used in combination with adjunct medical devices, such as introducers or sheaths. Preferably, such color-indications are color-coded to correspond to French size units that are used to indicate sizes in medical procedures.

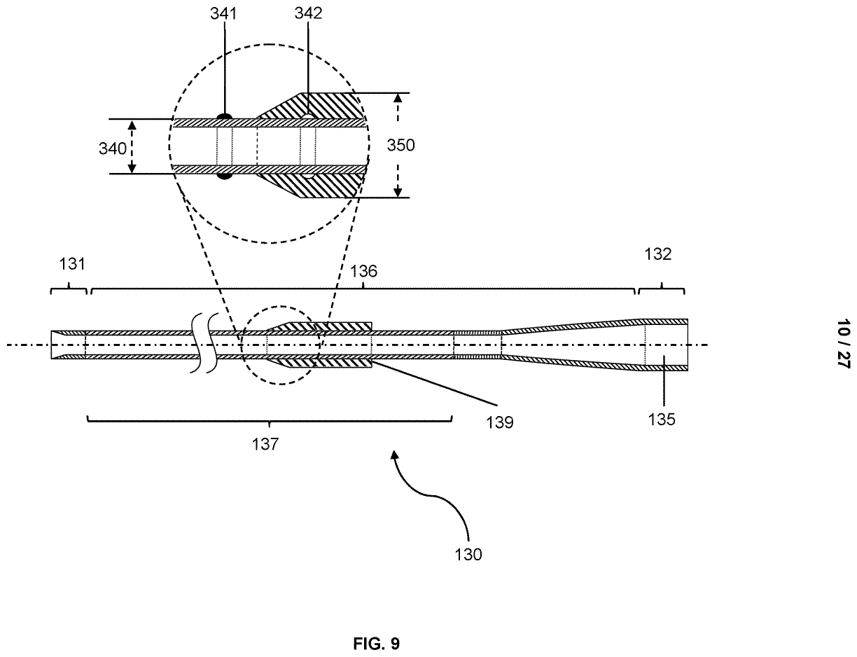

FIG. 9 illustrates a cross-sectional view of a sixth implementation of a self-centering adapter of the multi-functional catheter system. In its base construction, the sixth implementation of the adapter 130 is similar to the first implementation. However, the sixth implementation differs from the first implementation in that the adapter further comprises a cuff 139 coaxially received in a proximal or upper position on the adapter, that is configured for placement in a distal or lower position on the adapter and in turn can be located in a puncture site of a patient to be treated, thereby exerting a sealing force on an opening of the puncture site that enables hemostatic sealing. The cuff 139 is shown movably received around the outer surface of the adapter and formed of a different material than the proximal end 132, mid-section 136, cylindrical portion 137 and distal end 131. In FIG. 9, the outer diameter of the cuff 350 is shown smaller or equivalent to an outer diameter of the proximal end 132 of the adapter 130. However, depending on the typical diameter of a puncture site, and for ease of handling, the diameter of the cuff can also be of larger outer diameter than the outer diameter of the proximal end of the adapter 130. Further, the cuff 139 can exhibit the same or a different taper than a transition from the cylindrical portion 137 to a mid-section 136 or proximal end 132 of the adapter 130. In this example, the cuff tapers from the outer diameter 350 of the adapter to an outer diameter 340 of the cylindrical portion 137. The length of the cuff 139 can be smaller, equivalent to, or longer than a length of a wound canal, and the length of a cuff inserted into the puncture site by a treatment provider can be varied so as to variably adapt to a length present at a puncture site, as desired. In additional implementations, both cuff 139 and adapter 130 can be provided with complementary position-maintaining features 341 and 342, wherein superposition of these complementary position-maintaining features lock the position of the cuff with respect to the adapter and/or a puncture site. For example, a lockable cuff can be configured for placement in an upper, locked position on the adapter, and translated into one or more lower, locked positions on the adapter, that in turn can be located at different insertion depths of a puncture site of a patient, so as to enable various degrees of hemostatic sealing force. Alternatively, or complementary, the cuff can exert elastic properties that allow the cuff to contract or expand radially to facilitate both translation and locking onto the adapter at different positions and/or diameters of the adapter.

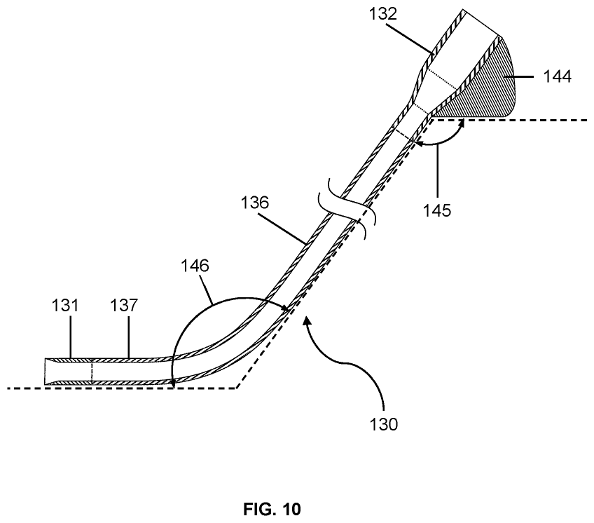

FIG. 10 illustrates a cross-sectional view of a seventh implementation of a self-centering adapter of the multi-functional catheter system. In its base construction, the seventh implementation of the adapter 130 is similar to the first implementation. However, in FIG. 10, the proximal end 132 of the adapter 130 includes a flange 144 that proceeds at an angle 145 from a mid-section 136 of the adapter to a proximal end 132 of the adapter that enables fixation of the adapter to a skin area of a patient to be treated. Further, a transition 137 proceeds at an angle 146 from a distal end 131 to a mid-section 136 of the shaft. The additional means of fixation aids a treatment provider in stabilizing the position or orientation of the adapter with respect to a blood vessel, wound canal, puncture site, or skin area of a patient to be treated, as well as with respect to co-administered diagnostic or therapeutic agents and/or adjunct medical devices. In complementary implementations, the adapter can comprise an additional flushing port, or alternatively, the proximal end can be shaped as a luer that enables coupling to an additional hub or manifold, that facilitates additional flushing and/or aspiration capability, as desired.

In summary, the self-centering adapter 130 of the multi-functional catheter system 100 is formed as a tubular member 135 that comprises a distal end 131, a mid-section 136 and a proximal end 132, wherein an outer diameter of the adapter at a distal end 131 is equivalent to an outer diameter of the distal catheter shaft 120; an inner diameter of the adapter at a distal end 131 is equivalent to an outer diameter of the proximal catheter extension shaft 140; an outer diameter of the adapter at a proximal end 132 exceeds an outer diameter of a distal end 151 of the hub 150, and an outer diameter of the mid-section 136 tapers between the outer diameters of the adapter at the distal 131 and proximal end 132.

In the various implementations, a contact surface 133 at the distal end 131 of the adapter 130 tapers inwards from an outer diameter of the adapter at a distal end 131 to an outer diameter of the proximal catheter extension shaft 140; and a contact surface 134 at the proximal end of the distal catheter shaft 120 tapers from an outer diameter of the distal catheter shaft 120 to an outer diameter of the proximal catheter extension shaft 140. In addition, the adapter 130 can include a transition 137 between the distal end 132 and the mid-section 136 of the adapter 130 that is cylindrical. A transition or overlapping region 137 between the proximal end 132 and the mid-section 136 of the adapter 130 can be formed from a malleable material and provided in a first shape that is elastically or plastically deformable to a second shape by an externally applied bending force thereby enabling shapeability of the adapter. Further, the transition or overlapping region 137 between the proximal end 132 and the mid-section 136 of the adapter 130 can be cylindrical and proceed at an angle 146 from the distal end to the mid-section of the adapter. The proximal end 131 can include a flange 144 that proceeds at an angle 145 from the mid-section 136 of the adapter 130 to the proximal end of the adapter 132 that enables fixation of the adapter to a skin area of a patient to be treated. In addition, an elastic modulus or hardness of the distal end 132 of the adapter 130 can be lower than an elastic modulus or hardness of the mid-section 136, 137 of the adapter, and an elastic modulus or hardness of the proximal end 131 of the adapter can exceed an elastic modulus or hardness of the mid-section 136, 137 of the adapter. The adapter may comprise length-adjustment features that allow the length of the adapter to variably adapt to one or more lengths present at the puncture site. In addition, a taper of the mid-section 136 can cover a range of diameters that allows the diameter of the adapter to variably adapt to one or more diameters present at the puncture site. Further, a taper of the mid-section 136 can cover a range of diameters that are provided color-indicated on a surface of the adapter. The adapter can comprise a spherical protrusion 138 positioned below and around a proximal end 131 of the adapter that during insertion of the adapter into a puncture site 402 exerts a sealing force on an opening of the puncture site 402, thereby enabling hemostatic sealing, or alternatively, the adapter can comprise a lockable cuff 139 coaxially received in an upper position on the adapter 130, and configured for placement in a lower position on the adapter that is located in a puncture site 402 of a patient to be treated, thereby exerting a sealing force on an opening of the puncture site that enables hemostatic sealing. Further, the adapter, and corresponding features present on the adapter, such as the cuff or the spherical protrusion can be provided with color-indications that are color-coded to correspond to French size units used to indicate sizes in medical procedures.

In general, the adapter can be formed from thermoplastic elastomers, which can include a combination of malleable and elastic elastomers, and the distal end 131, mid-section 136, 137 and proximal end 132 of the adapter 130 can be formed from the same or different thermoplastic elastomers, wherein the thermoplastic elastomers of the adapter 130 include at least one or more of acrylonitrile butadiene styrene, polytetrafluoroethylene, fluorinated ethylene propylene, polyvinyl chloride, polyamide, polyurethane, polyethylene, low density polypropylene, high density polypropylene, polyethylene imine, and silicone.

### Position-Maintaining and Sealing Features

FIG. 11 illustrates a cross-sectional view of a fluid-impermeable slideable seal that facilitates sliding of a proximal catheter extension shaft within a distal catheter shaft of the multi-functional catheter system. As shown in FIG. 11, the fluid-impermeable slideable seal includes an inner sealing annulus 143 that sealingly extends from an outer surface of the proximal catheter extension shaft 140 onto an inner surface of the distal catheter shaft 120. The distal catheter shaft 120 includes one or more positioning markers or elements, formed as annular bands, tubes or rings 125 embedded flush into the outer surface of the distal catheter shaft that in turn form one or more bump-like surfaces 223 on the inner surface of the distal catheter shaft. Because the height of the inner sealing annulus 143 protrudes onto the height of the bump-like surface 223 beneath the positioning marker 125, the bump-like surface 223 acts as a mechanical position-maintaining feature 123 that prevents the slideable seal from sliding proximally beyond the most proximal position of the one or more annular positioning marker bands, tubes or rings 125. In turn, when the proximal catheter extension shaft is in its most proximal position relative to the distal catheter shaft, the multifunctional catheter system is in fully extended state, and exhibits a maximum usable length. The sealing annulus 143 is preferably positioned in proximity to a distal end 142 of the proximal catheter extension shaft 140, and the positioning marker 125 of the mechanical position-maintaining feature 123 is preferably located in proximity to a proximal end of the distal catheter shaft 120. The inner sealing annulus provides a fluid-impermeable fitting that slideably connects a guidewire lumen 141 of the proximal catheter extension shaft with a guidewire lumen 124 of the distal catheter shaft to provide a single, continuous guidewire lumen that is usable-length-adjustable. In FIG. 11, the proximal end 142 of the proximal catheter extension shaft is formed as a tapered surface, that tapers from an inner diameter of the distal catheter shaft to an inner diameter of the proximal catheter extension shaft, so as to minimize any inner edges within the guide wire lumen, that in turn could impair movement of guidewires, adjunct medical devices, or transport of physiological, diagnostic or therapeutic agents, respectively.

FIG. 12 illustrates a cross-sectional view of a locking and position-maintaining feature between a proximal catheter extension shaft and a distal catheter shaft of the multi-functional catheter system. In FIG. 12, the proximal catheter extension shaft 140 is coaxially received within an inner guidewire lumen 124 of the distal catheter shaft 120. Shown in cross-section, several positioning markers or elements formed as annular bands, tubes or rings 125, 126, 127, positioned at regular intervals on an outer surface of the proximal catheter extension shaft protrude towards an inner surface of the distal catheter shaft 120. The distal catheter shaft 120 includes several annular striations 221, 222 embossed into an outer surface of the shaft, wherein the striations extend to an inner surface of the guidewire lumen 124 of the distal catheter shaft 120 that in turn, are complementary in shape and position to the annular positioning marker bands, tubes or rings 125, 126, 127 present on the outer surface of the proximal catheter extension shaft. Superposition of these complementary surface features lock the position of the proximal catheter extension shaft with respect to the distal catheter shaft and act as mechanical position-maintaining feature 121 and/or 122. In these implementations, the positioning markers or elements are preferably formed as annular bands, tubes or rings that preferably comprise radiopaque materials. However, concerning the individual form, composition and arrangement of these positioning markers, the markers are not limited as to the exact shape or disposition of materials, for example, the positioning markers can be formed from elastomeric or radiopaque, as well as colored or pigmented materials, or from a combination of elastomeric, radiopaque, colored or pigmented materials, that in turn are adhered, bonded, fused, welded, joined, or glued to, molded, embossed or embedded into, or layered onto or into a surface of the distal and proximal catheter shafts, respectively.

Application of an adequate longitudinal pulling or pushing force on the proximal catheter extension shaft while maintaining the position of the distal catheter shaft can overcome the forces that hold the positioning markers 125, 126, 127 of the proximal catheter extension shaft within the complementary striations 221, 222 of the distal catheter shaft. Thereby, the proximal catheter extension shaft can be incrementally translated with respect to the distal catheter shaft by incremental lengths equal to the distance between the positioning markers along the outer surface of the proximal catheter extension shaft and the complementary annular striations of the inner surface of the distal catheter shaft. The mechanical position-maintaining feature 121 is preferably positioned in proximity to a distal end or tip 110 of the distal catheter shaft 120, and the mechanical position-maintaining feature 122 is preferably positioned between the mechanical position-maintaining feature 121 and the mechanical position-maintaining and sealing feature 123. Because the multi-functional catheter system 100 includes at least two or more position-maintaining and sealing features 121, 122 and 123, translation of the proximal catheter extension shaft from a first position-maintaining feature 121 to a second 122 or consecutive position-maintaining feature 123 results in a respective length adjustment of the usable length of the catheter. The adjustable difference or increment in length of the catheter between a contracted and an extended usable length configuration is equivalent to the distance between the first, second and consecutive position-maintaining features. In these implementations, the position-maintaining features can be provided with additional color-indications that are color-coded to visually indicate corresponding usable-length increments to a treatment provider. Alternatively or complementary, the two or more position-maintaining features comprise radiopaque materials that angiographically indicate a position and/or increment of length between them. Further, movement of the proximal catheter extension shaft with respect to the distal catheter shaft from a locked to an un-locked position provide a haptic, tactile, or sensory signal to a treatment provider who is adjusting the usable length of a multi-functional catheter system. Accordingly, the usable length of the multi-functional catheter system 100 can be selected and adjusted by a treatment provider from a contracted to an extended state, and vice versa.

In summary, the position-maintaining features 121, 122, and 123 of the multi-functional catheter system 100 comprise at least one of:
a first set of positioning markers 125 positioned at regular intervals along the outer surface of the distal catheter shaft 120, each marker embedded flush within the shaft surface; the inner shaft surface below the marker protruding onto the sealing fitting 143 in order to impede sliding of the proximal catheter extension shaft 140 relative to the distal catheter shaft 120;
a second set of positioning markers 125, 126, 127 positioned at regular intervals along the outer surface of the proximal catheter extension shaft 140, and
a set of annular striations 221, 222 embossed into an outer surface of the shaft, wherein the striations extend to an inner surface of the guidewire lumen 124 of the distal catheter shaft 120 that in turn, are complementary in shape and position to the positioning markers 125, 126, 127, into one or more of which one or more of the positioning markers fits in order to impede sliding of the proximal catheter extension shaft 140 relative to the distal catheter shaft 120,
wherein the set of annular striations 221, 222 patterned along the external surface of the distal catheter shaft facilitate manual translation of the proximal catheter extension shaft 140 relative to the distal catheter shaft 120,
wherein the regular intervals between the position-maintaining features determine an amount of usable-length-adjustment of the multi-functional catheter system,
wherein the position-maintaining features are provided with color-indications that are color-coded to visually indicate usable-length increments to a treatment provider, and further
wherein the position-maintaining features are formed from a combination of elastomeric, radiopaque, colored or pigmented materials.

### Torqueing Features

FIG. 13A-C illustrate cross-sectional views of several implementations of torqueing features of the proximal and distal shafts of the multi-functional catheter system. FIG. 13C shows, in cross-section, a distal catheter shaft 120 of the multi-functional catheter system 100. The distal catheter shaft as shown includes several orientation maintaining features 224 and 225, located at cross-section planes A-A and B-B, respectively, that protrude into the guidewire lumen 124 of the distal catheter shaft that in turn is intended to receive the proximal catheter extension shaft 140. FIG. 13 A provides a front-view of the cross-section A-A of a first implementation of an orientation maintaining feature 224. In FIG. 13A, the distal catheter shaft 120 includes one or more orientation maintaining features 224, formed as concave indentations on the shaft surface and oriented at a 120° angle with respect to the center of the guidewire lumen 124. A proximal catheter extension shaft 140 is shown received within the guidewire lumen 124, and includes one or more complementary orientation maintaining features 226, formed as concave indentations on the shaft surface and oriented at a 120° angle with respect to the center of the guidewire lumen 141. FIG. 13B provides a front-view of the cross-section B-B of a second implementation of an orientation maintaining feature 225, that differs from the first implementation in that two orientation maintaining features 225 of the distal catheter shaft 120 are oriented at a 180° angle with respect to the center of the guidewire lumen 124, and two orientation maintaining features 227 of the proximal catheter shaft 140 are oriented at a 180° angle with respect to the center of the guidewire lumen 141. When the orientation maintaining feature 224 (225) is moved out of the position-maintaining feature 226 (227) by longitudinal translation of the proximal catheter extension shaft 140 with respect to the distal catheter shaft 120, both shafts can be freely rotated with respect to another. Superposition of the complementary orientation maintaining features 224 and 226 (or 225 and 227) lock the orientation of the proximal catheter extension shaft 140 with respect to the distal catheter shaft 120 and act as orientation maintaining or torqueing feature. The length 228 and/or 229 of the orientation maintaining features 224, 226 and/or 225, 227 along a shaft of the multi-functional catheter system can be varied, so as to vary a lockable length of the orientation maintaining features, when superimposed onto each other. For example, the length 228, 229 of the features can extend a relatively short section of the shafts, as shown, or be continued along a considerable length of the distal and proximal shafts. Alternatively, the orientation-maintaining feature may extend along the complete length of one of the distal and proximal shafts or both, so as to enable maintaining orientation at any relative position of one shaft to the other. As a result of this arrangement, the application of torque or rotational force exerted by a treatment provider on a proximal catheter extension shaft 140 can be directly transmitted to a distal catheter shaft 120. Because the catheter tip 110 is fixedly connected to the distal catheter shaft, the application of torque on the proximal catheter extension shaft 140 is thereby transmitted to the catheter tip 110 via the distal catheter shaft, enabling enhanced steerability of the system within a vasculature of a patient to be treated.

In summary, the distal catheter shaft 120 and proximal catheter extension shaft of the multi-functional catheter system of the current disclosure can include two or more orientation maintaining features 224, 226 and/or 225, 227 that reversibly lock or unlock an orientation of the proximal catheter extension shaft 140 with respect to the distal catheter shaft 120, and in turn enable the transmission of torque or rotational force by a treatment provider to a catheter tip, thereby facilitating enhanced steerability of the system within a vasculature of a patient to be treated.

### Catheter Tip and Guidewire Lumen Configuration

FIG. 14 illustrates a cross-sectional view of a single-lumen catheter tip of the multi-functional catheter system, as a first implementation. In FIG. 14, a distal catheter shaft 120 of the multi-functional catheter system extends into a catheter tip 110, and a guidewire lumen 124 of the distal catheter shaft 120 extends to an opening or guidewire port at the catheter tip 110 that provides for insertion, transport, removal and/or exchange of a guidewire 101. The catheter tip 110 further includes a radiopaque marker 111 adhered to a surface of the tip, and comprises a soft distal end 103, that enables the atraumatic insertion into a puncture site of a patient to be treated. Because each catheter shaft 120 and 140 of the multi-functional catheter system includes one or more guidewire lumen 124, 141, that are joined together at a sealing fitting 143, and extend from an opening or guidewire port at the proximal end of the hub 152 to the opening or guidewire port at the catheter 110 tip, the first implementation of the catheter tip represents a single-lumen guidewire configuration, that facilitates the operation of the multi-functional catheter system in OTW mode. In this implementation, an elastic modulus of the catheter tip 110 is equivalent or smaller than an elastic modulus of the distal catheter shaft 120. In comparison, an elastic modulus or hardness of the radiopaque material of the catheter tip 110 can at least be equivalent or higher than an elastic modulus of the elastomeric material of the tip.

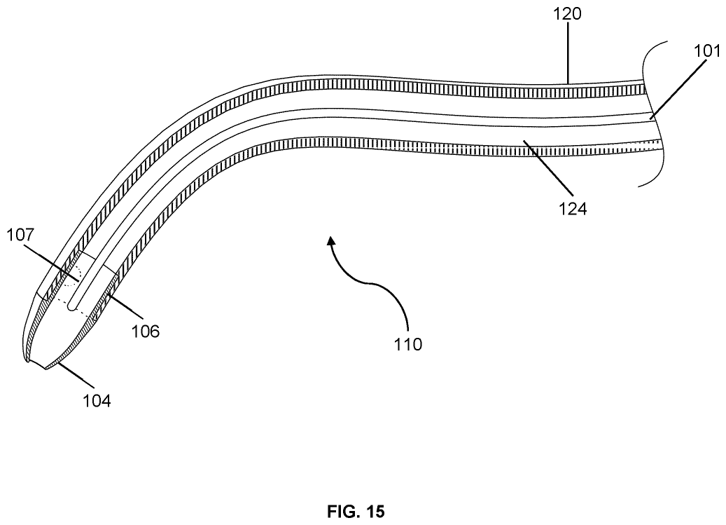

FIG. 15 illustrates a cross-sectional view of a single-lumen catheter tip of the multi-functional catheter system, as a second implementation. In FIG. 15, the second implementation differs from the first implementation of FIG. 14, in that the catheter tip 110 comprises a rigid distal end 104. The distal end 104 is formed as a conical tip that includes a mantle surface 106 having an outer diameter smaller than an outer diameter of the distal catheter shaft, and circular openings 107, that allow the tip to be fixedly embedded or bonded to an inner surface at a distal end of the distal catheter shaft 120. An outer diameter of the distal end 104 tapers from an outer diameter at the distal end of the shaft 120, and an inner diameter of the distal end 104 tapers from an inner diameter of the distal shaft 120 to a smaller inner diameter at an opening of the distal end of the tip. In this implementation, the elastic modulus or hardness of the catheter tip 110 (that includes the rigid distal end 104) can exceed a range of elastic moduli or hardness present in the vasculature thereby enabling crossing capability of the tip. Similar to the first implementation, the catheter tip can include a radiopaque marker, or be entirely formed from a radiopaque material, which includes metals, such as stainless steel. Alternatively, the catheter tip can include rigid polymers, such as PEEK, that have radiopaque materials dispersed therein.

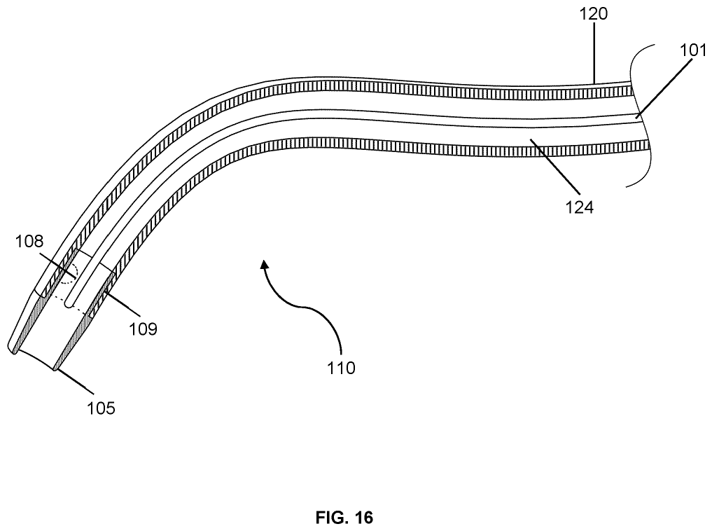

FIG. 16 illustrates a cross-sectional view of a single-lumen catheter tip of the multi-functional catheter system, as a third implementation. In FIG. 16, the third implementation differs from the second implementation of FIG. 15, in that the distal end 105 of the catheter tip 110 is formed as a tapered tip that includes a mantle surface 109 having an outer diameter smaller than an outer diameter of the distal catheter shaft, and circular openings 108, that allow the tip to be fixedly embedded or bonded to an inner surface at a distal end of the distal catheter shaft 120. In comparison to the second implementation, the inner diameter of the distal end 105 is equivalent to an inner diameter of the distal catheter shaft 120.

In the various implementations of the catheter tip 110 of the multi-functional catheter system 100, the catheter tip can be formed from an elastomeric material, and provided in a straight, shaped, or shapeable configuration. For example, the elastomeric material of the catheter tip 110 can be provided in a first shape that is elastically or plastically deformable to a second shape by insertion or removal of a guidewire having a higher stiffness than the elastomeric material of the tip, thereby enabling shapeability of the catheter tip. The transition between the first and the second shape of the catheter tip 110 can occur prior to or during a procedure, and enables steering of the catheter tip in a vasculature of a patient. In the various implementations, an elastic modulus or hardness of the catheter tip 110 can be configured to not exceed a range of elastic moduli or hardness present in the vasculature thereby enabling atraumatic maneuvering capability of the tip. Alternatively, an elastic modulus or hardness of the catheter tip 110 can exceed a range of elastic moduli or hardness present in the vasculature thereby enabling crossing capability of the tip. Further, the catheter tip 110 can be formed from a combination of elastomeric and radiopaque materials, and an elastic modulus of the radiopaque material of the catheter tip 110 can at least be equivalent or higher than an elastic modulus of the elastomeric material of the tip or the shaft. The catheter tip implementations share in common, that a guide wire lumen 141, 124 of the proximal and distal catheter shaft is extended into a single lumen 129 of the catheter tip 110.

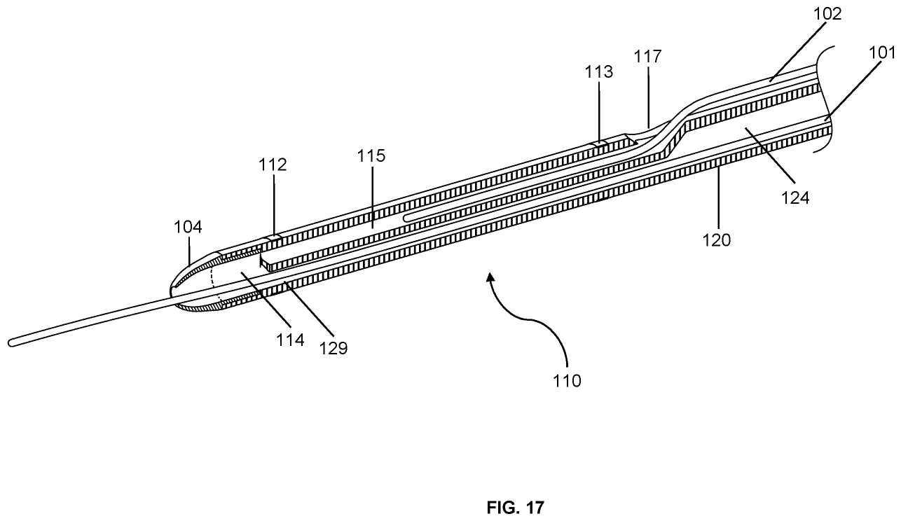

FIG. 17 illustrates a cross-sectional view of a dual-lumen catheter tip of the multi-functional catheter system, as a fourth implementation. In FIG. 17, the catheter tip 110 comprises a dual-lumen configuration represented by (i) guidewire lumen 129 and (ii) guidewire lumen 115. A length section of the catheter tip 110 that contains the two guidewire lumens 129 and 115 is delineated by two annular radiopaque markers 112 and 113 embedded into an outer surface of the distal shaft 120, that indicate resting positions for one or more guidewires 101 and 102 that can be positioned simultaneously or consecutively within the dual-lumen section of the catheter tip. The single guidewire lumen 124 of the distal catheter shaft extends through the guidewire lumen 129 of the dual-lumen section, which in turn extends through a space 114 to an opening at the distal end 104 of the catheter tip 110. The second guidewire lumen 115 extends from an opening at the distal end 104 through a space 114 of the catheter tip 100 and through the dual-lumen section, to an opening 117 present on a lateral surface of the distal catheter shaft 120, which is located proximal to the radiopaque marker 113. The opening or guidewire port 117 includes a ramp-like feature that allows the guidewire 102 to be deflected out of the opening and away from the lateral surface of the distal catheter shaft, so that the guidewire 102 can be maintained outside of the distal catheter shaft once it has passed a proximal position indicated by radiopaque marker 113. The dual lumen 129, 115 of the catheter tip 110 enables the simultaneous positioning of one or more guidewires 101, 102 in the one or more resting positions in the catheter tip 110, thereby enabling the simultaneous or consecutive use of the guidewires without loss of access to a treatment site or requiring the exchange of the one or more guidewires. Thereby, the fourth implementation of the catheter tip 110 represents a dual-lumen guidewire configuration that facilitates the simultaneous or consecutive operation of the multi-functional catheter system in RX or OTW guidewire exchange mode. As a specific technical feature, the positioning of a distal end of the guidewire 102 at the resting position indicated by radiopaque marker 112 temporarily blocks the guidewire lumen 115, thereby enabling the focal delivery of physiological, therapeutic and diagnostic fluids across the guidewire lumen 129 (when the guidewire 101 is not positioned in the guidewire lumen 129), and ensuring minimal risk of leaking physiological, diagnostic or therapeutic fluids and/or agents through the opening or guidewire port 117.

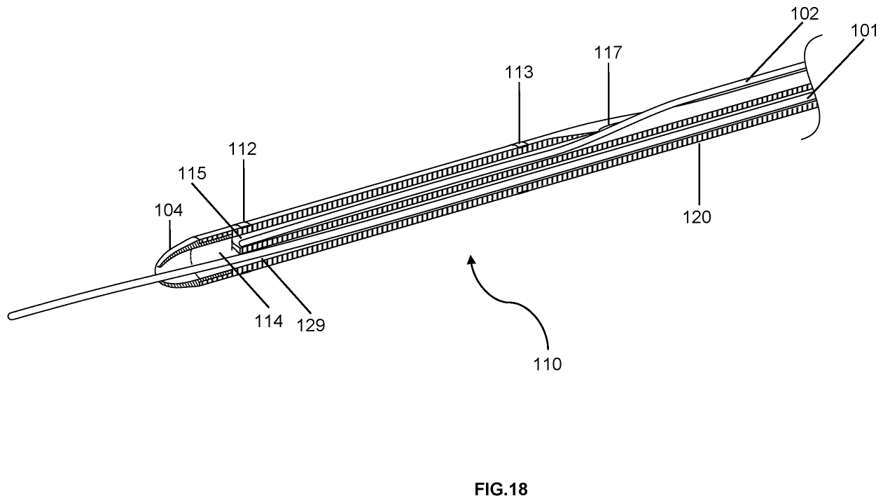

FIG. 18 illustrates a cross-sectional view of a dual-lumen catheter tip of the multi-functional catheter system, as a fifth implementation. In FIG. 18, the fifth implementation of the catheter tip differs from the fourth implementation, in that the opening or guidewire port 117 does not contain ramp-like features through which a guidewire would be deflected at an angle away from the surface of the distal catheter shaft 120. In this example, the outer diameter of the distal catheter shaft 120, starting from a position on the shaft indicated by radiopaque marker 113, tapers in proximal direction to a smaller outer diameter. Thereby, the proximal section of the distal catheter shaft 120 can exhibit a smaller outer diameter as compared to the fourth implementation. Because the opening 117 does not contain a ramp-like feature that would otherwise deflect a guidewire 102 away from the lateral surface of the distal catheter shaft, the guidewire can proceed directly parallel and in close proximity to the shaft surface, resulting in a reduced crossing profile of the multi-functional catheter system.

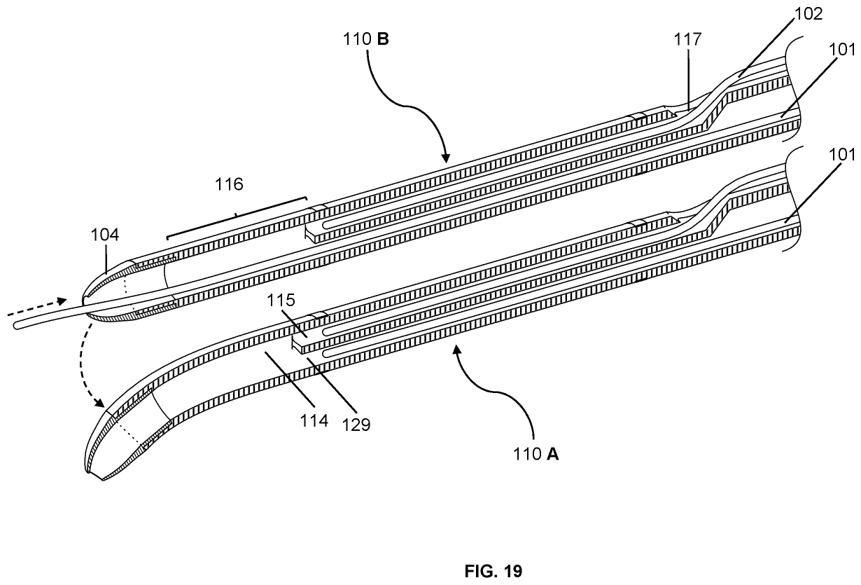

FIG. 19 illustrates shaping of a dual-lumen catheter tip of the multi-functional catheter system, as a sixth implementation. In FIG. 19, the dual-lumen configured catheter tip 110 is shown in a pre-shaped configuration 110 A and in a straightened configuration 110 B. An elastomeric material of the catheter tip 110 is provided in a first shape 110 A, that is elastically or plastically deformable to a second shape 110 B by insertion or removal of a guidewire 101 having a higher stiffness than the elastomeric material thereby enabling shapeability of the catheter tip. The pre-shaped configuration 110 A is straightened to configuration 110 B by insertion of one or more guidewires 101 or 102 through an opening at the distal end 104 of the catheter tip 110. Pulling the guidewire 101 in proximal direction, as indicated by dashed arrows, the straightened catheter tip 110 B reassumes its pre-shaped state 110 A. In this example, the dual-lumen section 129, 115 of the catheter tip 110 extends into a single-lumen section 116 that enables containing and mixing of physiological, therapeutic and diagnostic fluids.

In these implementations of the catheter tip 110 of the multi-functional catheter system 100, the guide wire lumen 141, 124 of the proximal and distal catheter shaft can extend into a dual lumen 129, 115 of the catheter tip 110, and one of the two lumens can extend from the catheter tip 110 into a guidewire port 117 present on a lateral surface of the catheter shaft. The dual lumen 129, 115 of the catheter tip 110 in turn can extend into a single lumen 116 that enables containing and mixing of physiological, therapeutic and diagnostic fluids. A position of the lumen is indicated by one or more radiopaque materials 112, 113 that in turn indicate one or more resting positions of one or more guidewires 101, 102. The dual lumen 129, 115 of the catheter tip 110 enables the simultaneous use of one or more guidewires 101, 102 in a rapid-exchange (RX) and/or over-the-wire (OTW) configuration. Further, the dual lumen 129, 115 of the catheter tip 110 enables the simultaneous positioning of one or more guidewires 101, 102 in the one or more resting positions in the catheter tip 110, thereby enabling the simultaneous or consecutive use of the guidewires without loss of access to a treatment site and/or requiring the exchange of the one or more guidewires. In addition, the positioning of one of the one or more guidewires 101, 102 in one of the one or more resting positions in the catheter tip 110 temporarily blocks the guidewire lumen 115, thereby enabling the focal delivery of physiological, therapeutic and diagnostic fluids across the guidewire lumen 129.

### Dimensions and Usable-Length-Adjustment

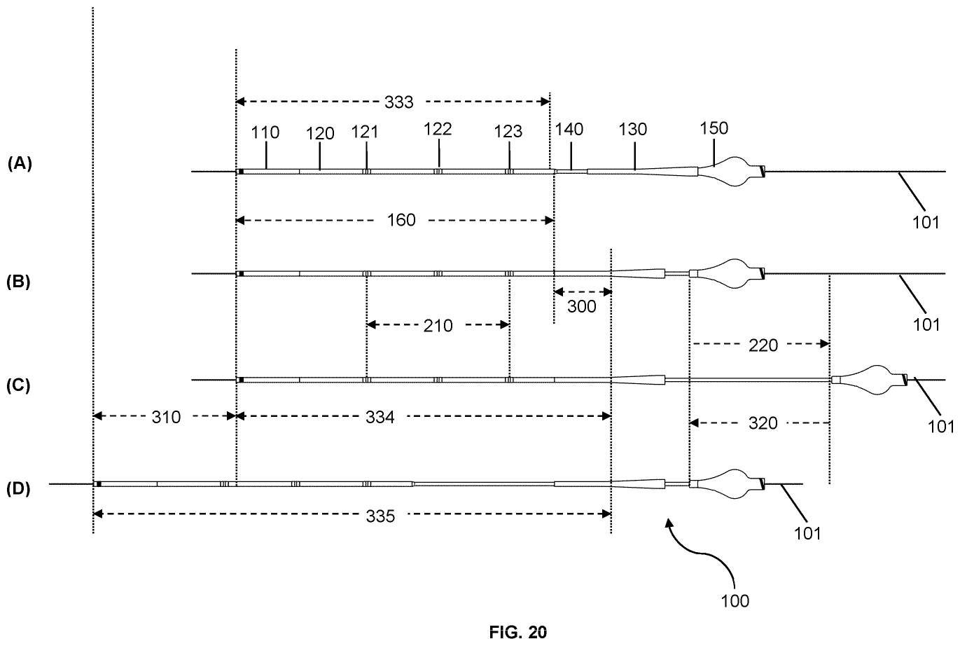

FIG. 20A-D illustrate cross-lateral views representing four consecutive configurational stages A-D for operating the self-centering adapter and adjusting the usable length of the catheter system, as several embodiments.

In FIG. 20A, the multi-functional catheter system 100 is shown in a fully contracted state, wherein the adapter 130 is coupled to a distal end of the hub 150 in a first or resting position, such that the proximal catheter extension shaft 140 is kink protected. The proximal catheter extension shaft 140 is slideably inserted into the distal catheter shaft 120 by a treatment provider, and arrested at position-maintaining feature 121 of the distal catheter shaft 120. In this state, the multi-functional catheter system exhibits a first, minimum usable length 333, which is slightly shorter than the length of the distal catheter shaft 160, so that a proximal end of the distal catheter shaft can be maintained proximal to a puncture site and thereby, outside of a patient.

In FIG. 20B, the multi-functional catheter system 100 is shown in a fully contracted state, wherein the adapter 130 is coupled to a proximal end of the distal catheter shaft in a second or docking position-while the position of the distal catheter shaft 120 is maintained by a treatment provider-such that a resulting transition between a diameter of the adapter and a diameter of the distal catheter shaft is edge-and seamless. In this state, the multi-functional catheter system exhibits a second usable length 334 that is longer than the length of the distal catheter shaft 160, as the adapter 130 has been coupled to the proximal end of the distal catheter shaft 120, and can thus be inserted together with the distal catheter shaft until the adapter is positioned in a puncture site of a patient. The coupling of the adapter to the distal shaft therefore extends the first usable length 333 to a second usable length 334, by length increments largely defined through a length aspect 300 of the adapter.

In FIG. 20C, the multi-functional catheter system 100 is shown in a fully extended state, wherein the position of the adapter 130, together with the distal catheter shaft is maintained by a treatment provider, while the proximal catheter extension shaft has been moved proximally by a distance 220, and arrested into the third position-maintaining feature 123. In this example, the distance 220 is equivalent to a distance or length 210 between the first and third position-maintaining feature 121, and 123. Thereby, the multi-functional catheter system 100 is selectively length-adjusted by a treatment provider to a third usable length 335 that is available for insertion into a patient to be treated.

In FIG. 20D, the multi-functional catheter system 100 is shown in a fully extended state, wherein the position of the adapter 130 is maintained by a treatment provider, and the proximal catheter extension shaft has been moved distally by a distance 320 while being locked into the third position-maintaining feature 132. Thereby, the distal catheter shaft is extended distally by a distance 310 that is equivalent to the distance 320, which in turn is equivalent to the distance or length 210 between the first and third position-maintaining feature 121, and 123.

Because the multi-functional catheter system contains two or more position-maintaining features 121, 122, 123, the usable length of the catheter system can be adjusted in incremental steps defined by the individual distances or lengths present between the two or more position-maintaining features. As a result of this arrangement, the usable length of the multi-functional catheter system can be selected and adjusted by a treatment provider over a set of lengths prior to and during medical procedures. As a specific technical effect of using the self-centering adapter in combination with the multi-functional catheter system of the current disclosure, the set or number of usable lengths that is available for treatment is larger than the set of usable lengths that would otherwise be available in conventional length-adjustable catheters: Coupling of the adapter 130 to the distal shaft 120 extends a first usable length 333 to a second usable length 334, by an amount determined through a length 300 of the adapter.

In the following, methods of using and operating the multi-functional catheter system will be described.

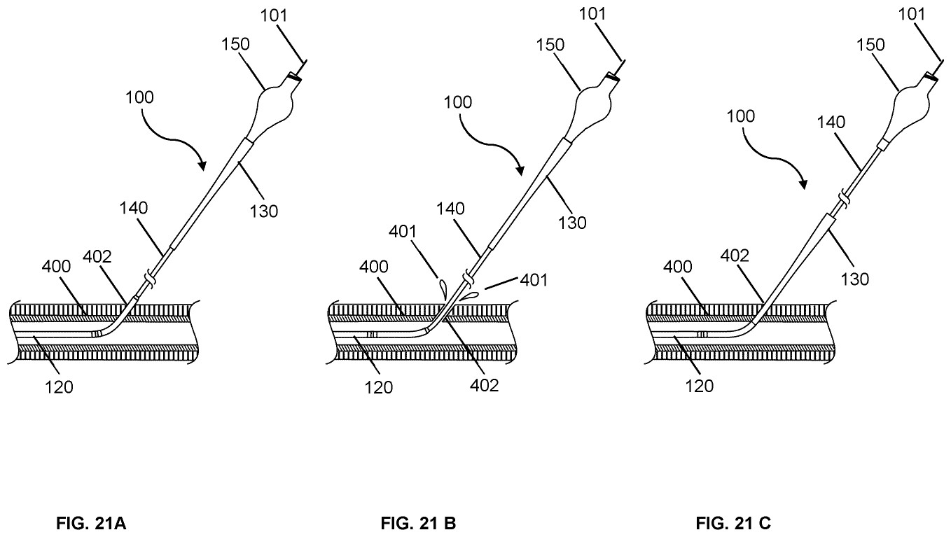

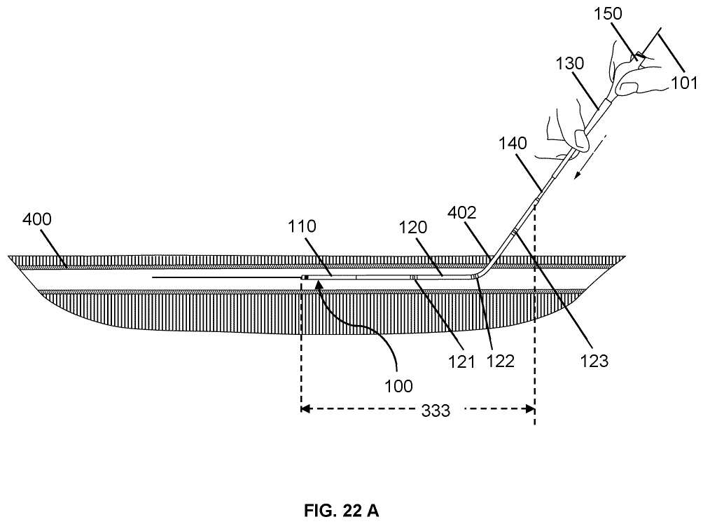

FIG. 21A-C illustrate cross-lateral views representing the various effects of placing a multi-functional catheter system into a puncture site of a patient in the absence or presence of the self-centering adapter. In these implementations, the multi-functional catheter system is deployed without the use of an introducer or sheath.

In FIG. 21A, the multi-functional catheter system 100 is shown inserted through a puncture site 402 and into a vessel 400 of a patient to be treated without the use of an introducer or sheath. The outer diameter of the proximal end of the distal catheter shaft is commensurate with an outer diameter of the puncture site 402, which is sufficient to hemostatically seal the puncture site. This infers that the distal catheter shaft of the multi-functional catheter system can be used in a sheath-less mode of operation, as long as the proximal end of the distal catheter shaft is maintained outside of a patient by a treatment provider. In FIG. 21A, the adapter 130 is still maintained at a distal end of the hub 150 in a first or resting position, so as to enable kink-protection of the proximal catheter extension shaft.

In FIG. 21B, the multi-functional catheter system 100 is shown inserted through a puncture site 402 and into a vessel 400 of a patient to be treated without the use of an introducer or sheath. However, in comparison to FIG. 21A, the proximal end of the distal catheter shaft has been moved distally beyond the puncture site 402. Because the proximal catheter shaft 140 is smaller in outer diameter than the outer diameter the distal catheter shaft, a dimensional gap is formed between proximal shaft 140 and the puncture site 402. As a result, blood 401 can leak from the puncture site 402 due to insufficient hemostatic sealing. This undesirable effect is beneficially prevented by using a self-centering adapter 130 of the current disclosure, as follows:

In FIG. 21C, the multi-functional catheter system 100 is shown inserted through a puncture site 402 and into a vessel 400 of a patient to be treated without the use of an introducer or sheath. The adapter 130 has been coupled to a proximal end 134 of the distal catheter shaft 120 in a second or docking position by a treatment provider, such that a resulting transition between a diameter of the adapter 130 and a diameter of the distal catheter shaft 120 is edge-and seamless. In this docking position, when the adapter has been coupled to the distal catheter shaft, the distal catheter shaft can be inserted in unison with the adapter into the puncture site. Accordingly, in FIG. 21C, in the second position, the adapter 130 has been placed in an unprotected puncture site 402 of a patient to be treated, such that the multi-functional catheter system, including the proximal catheter extension shaft, is movably centered in the then protected puncture site without direct contact thereto. Due to the tapered design of the adapter, the outer diameter of the adapter can be variably adapted to an outer diameter of the puncture site. Because the outer diameter of the adapter is equivalent or larger than the outer diameter of the distal catheter shaft, which in turn is commensurate to an outer diameter of the puncture site, the proximal catheter extension shaft can be slideably inserted through the puncture site without causing hemorrhage. As a result of this specific arrangement, the adapter thereby enables the atraumatic insertion, maneuvering and removal of the proximal catheter shaft of the multi-functional catheter system without the need of an additional introducer or sheath.

Because both the distal catheter shaft 120 of the multi-functional catheter system 100 and the proximal catheter extension shaft 140 can be inserted without an additional introducer or sheath into a puncture site 402 of a patient to be treated, when the self-centering adapter has been coupled to the proximal end of the distal shaft 120, the self-centering adapter 130 of the current disclosure therefore enables the atraumatic insertion, maneuvering and removal of the multi-functional catheter system 100 without the need of an additional introducer or sheath.

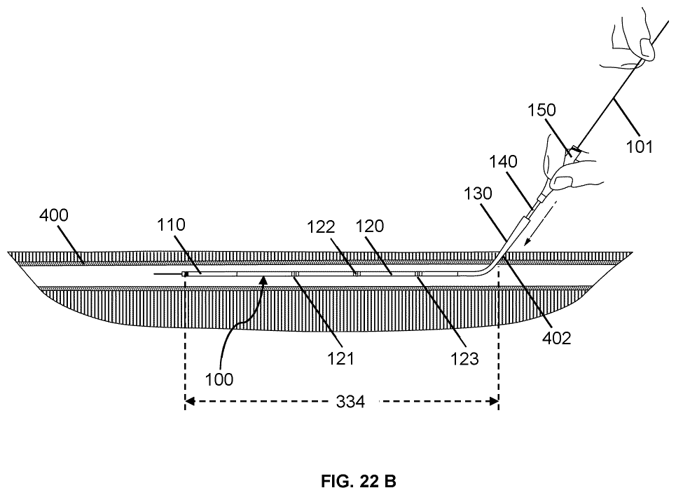

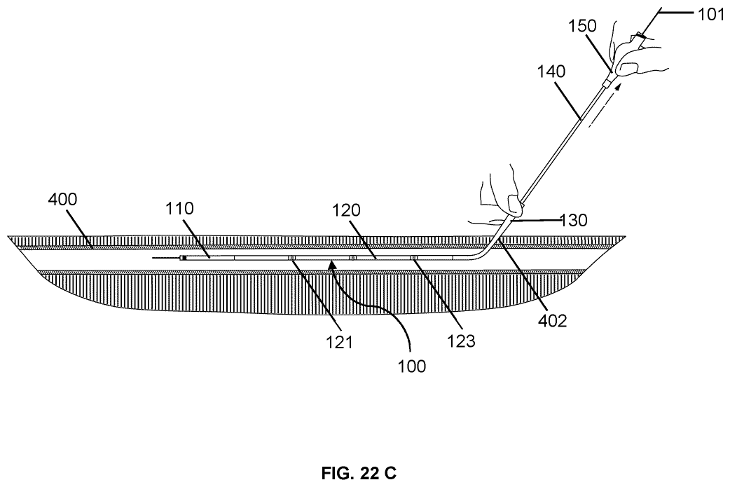

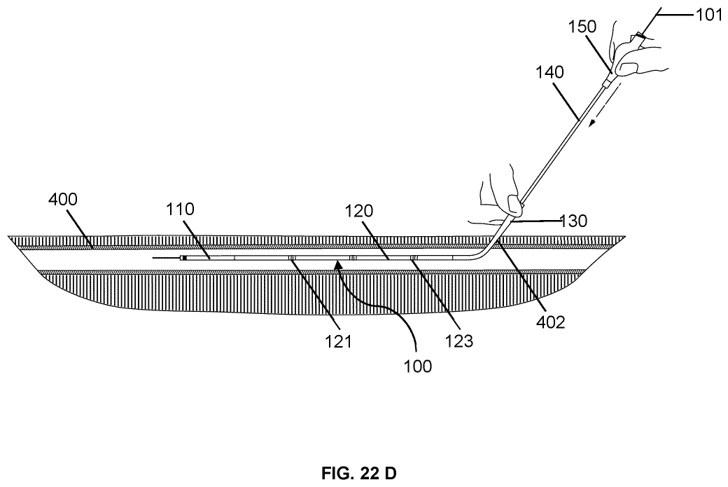

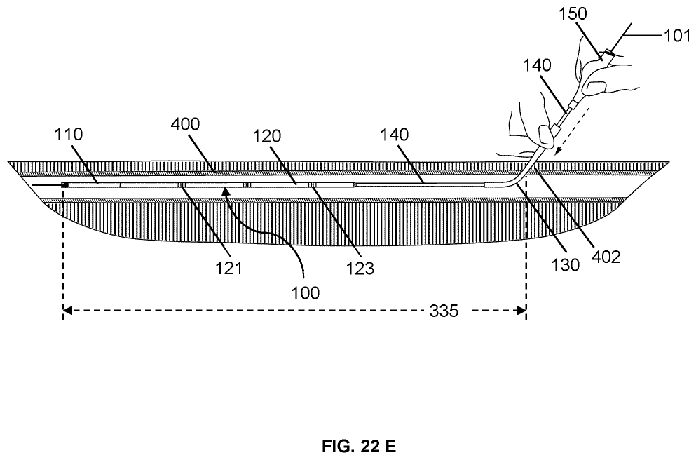

FIG. 22A-E illustrate a series of steps carried out by a treatment provider, using a multi-functional catheter system configured for usable-length extension in combination with an integrated, self-centering adapter to access multiple treatment sites within a patient's vasculature.

In FIG. 22A, the multi-functional catheter system 100 is in a fully contracted state having a first, minimum usable length. The instrument is shown inserted over a guidewire 101 through a puncture site 402 and into a vessel 400 of a patient to be treated-without the use of an introducer or sheath. In this state, the distal end of the proximal catheter extension shaft 140 is arrested in the first position-maintaining feature 121, whereas the proximal end of the distal catheter shaft still remains proximal to the puncture site and outside of the patient.

As shown in FIG. 22A, the indwelling length of the multi-functional catheter spans a distance from the distal end of the catheter tip 110 to the puncture site 402 that is smaller than the first, or minimum usable length 333 of the multi-functional catheter. When the first treatment site is located at a distance from the puncture site that is smaller or equivalent to a first, minimum usable length 333 of the multi-functional catheter, the proximal end of the distal catheter shaft is maintained outside of the patient, and the treatment provider can perform a first treatment at a treatment site without using the self-centering adapter. When a second treatment site is located at a distance from the puncture site that is larger than the first, minimum usable length, a treatment provider can undergo the following series of steps in extending the usable length of the multifunctional catheter in combination with the self-centering adapter, to access consecutive treatment sites within a patient's vasculature:

As a first step, a treatment provider maintains the position of the proximal catheter extension shaft by gripping at the hub 150, and moving the integrated adapter 130 that is slideably received on the proximal shaft from a first or resting position, wherein the adapter is coupled to a distal end of the hub 150, to a second or docking position, wherein the adapter is coupled to a proximal end of the distal catheter shaft 120.

In FIG. 22B, as a second step, the treatment provider maintains the position of the guidewire 101, and advances adapter 130, proximal catheter extension shaft 120, and distal catheter shaft 120 in unison distally, until the adapter 130 is positioned within the then protected puncture site 402, and the catheter tip 110 is positioned at a second or consecutive treatment site, where the treatment provider then can utilize the catheter system at the second usable length 334 to carry out a second or consecutive treatment, as desired.

In FIG. 22C, as a third step, the treatment provider maintains the position of the adapter 130 within the puncture site, variably adapting diameter and/or insertion length of the adapter to the puncture site, so as to enable various degrees of hemostatic sealing force and/or pressure, while retracting the proximal catheter extension shaft 140 by gripping hub 150 and pulling the shaft 140 proximally, such that the distal end of the shaft 140 is moved out of the first position-maintaining feature 121 and arrested in the third position-maintaining feature 123. As a result, the multi-functional catheter system 100 is now in a fully extended state, and exhibits a third, maximum usable length that is ready for insertion into the patient to be treated.

In FIG. 22D shows the beginning of the fourth step. In FIG. 21D, the treatment provider maintains the position of the adapter 130 within the puncture site 402 by stabilizing the adapter 130 manually, or by fixing the adapter to a skin area of a patient, maintaining control over diameter, insertion length and hemostatic sealing force and/or pressure of the adapter on the puncture site, while advancing the proximal catheter extension shaft 120 and distal catheter shaft 120 in unison distally through the adapter 130 that is positioned in the protected puncture site 402, by pushing the hub 150.

In FIG. 22E shows the completion of the fourth step. In FIG. 21E, the multi-functional catheter system 100 is now in a fully extended state having a third, maximum usable length 335. At the end of the fourth step, the treatment provider still maintains the position of the adapter 130 within the protected puncture site by stabilizing the adapter, maintaining control over diameter, insertion length and hemostatic sealing force and/or pressure of the adapter on the puncture site, while advancing the proximal catheter extension shaft 120 and distal catheter shaft 120, until the catheter tip 110 is positioned at a second or consecutive treatment site, where the treatment provider then can utilize the catheter system at the third, maximum usable length to carry out a third or consecutive treatment, as desired. Near completion of the treatment, when the proximal catheter extension shaft is in an extended state, and the adapter still positioned in the puncture site of a patient to be treated, proximal retraction of the proximal catheter extension shaft by a treatment provider docks or couples the self-centering adapter onto the proximal end of the distal catheter shaft. In this state, the multi-functional catheter system, including proximal catheter extension shaft and adapter can be atraumatically removed from a patient without requiring an additional introducer or sheath.

The foregoing description, for purposes of explanation, refers to specific nomenclature to provide a thorough understanding of the invention. However, it will be apparent to one skilled in the art that the specific details are not required in order to practice the invention. The foregoing descriptions of specific implementations of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and practical applications, to thereby enable others skilled in the art to best utilize the invention and various implementations with various modifications as suitable for the particular uses contemplated. It is intended that the scope of the invention be defined by the following claims and their equivalent.

1. A multi-functional catheter system (100) comprising:
a hub (150);
a guidewire lumen (124, 141);
a proximal catheter extension shaft (140);
a distal catheter shaft (120);
a catheter tip (110);
two or more guidewire ports (152); and
a self-centering adapter (130) formed as a tubular member (135), that comprises distal end (131), a mid-section (136) and a proximal end (132);
wherein the adapter (130) is slideably received on the proximal catheter extension shaft (140) and configured to couple to a distal end (151) of the hub (150) in a first or resting position, such that the proximal catheter extension shaft (140) is kink protected;
wherein the adapter (130) is further configured to couple to a proximal end (134) of the distal catheter shaft (120) in a second or docking position, such that a resulting transition between a diameter of the adapter and a diameter of the distal catheter shaft is edge-and seamless;
wherein in the second position, the adapter (130) is configured for variable placement in an unprotected puncture site (402) of a patient to be treated, such that the proximal catheter extension shaft (140) is movably centered in, and thereby, protects the puncture site without direct contact thereto, and wherein the adapter (130) thereby enables the atraumatic insertion, maneuvering and removal of the proximal catheter extension shaft (140) of the multi-functional catheter system without the need of an additional introducer.

2. (canceled)

3. The multi-functional catheter system according to claim 1, wherein
an outer diameter of the adapter at a distal end (131) is equivalent to an outer diameter of the distal catheter shaft (120);
an inner diameter of the adapter at a distal end (131) is equivalent to an outer diameter of the proximal catheter extension shaft (140);
an outer diameter of the adapter at a proximal end (132) exceeds an outer diameter of a distal end (151) of the hub (150), and
an outer diameter of the mid-section (136) tapers between the outer diameters of the adapter at the distal (131) and proximal end (132).

4. The multi-functional catheter system according to claim 1, wherein a contact surface (134) at the proximal end of the distal catheter shaft (120) tapers from an outer diameter of the distal catheter shaft (120) to an outer diameter of the proximal catheter extension shaft (140).

5. The multi-functional catheter system according to claim 1, wherein a contact surface (133) at the distal end (131) of the adapter (130) tapers inwards from an outer diameter of the adapter at a distal end (131) to an outer diameter of the proximal catheter extension shaft (140).

6. The multi-functional catheter system according to claim 1, wherein a transition (137) between the distal end (131) and the mid-section (136) of the adapter (130) is cylindrical.

7. The multi-functional catheter system according to claim 1, wherein a transition (137) between the proximal end (132) and the mid-section (136) of the adapter (130) is formed from a malleable material and provided in a first shape, that is elastically or plastically deformable to a second shape by an externally applied bending force thereby enabling shapeability of the adapter.

8. The multi-functional catheter system according to claim 1, wherein a transition (137) between the proximal end (132) and the mid-section (136) of the adapter is cylindrical and proceeds at an angle (146) from the distal end (131) to the mid-section of the adapter.

9. The multi-functional catheter system according to claim 1, wherein the proximal end (132) includes a flange (144) that proceeds at an angle (145) from the mid-section of the adapter to the proximal end of the adapter that enables fixation of the adapter to a skin area of a patient to be treated.

10. The multi-functional catheter system according to claim 1, wherein an elastic modulus or hardness of the distal end (131) of the adapter (130) is lower than an elastic modulus or hardness of the mid-section (136) of the adapter, and wherein an elastic modulus or hardness of the proximal end (132) of the adapter exceeds an elastic modulus or hardness of the mid-section of the adapter.

11. The multi-functional catheter system according to claim 1, wherein the adapter comprises length-adjustment features that allow the length of the adapter to variably adapt to one or more lengths present at the puncture site.

12. The multi-functional catheter system according to claim 3, wherein a taper of the mid-section (136) covers a range of diameters that allow the diameter of the adapter to variably adapt to one or more diameters present at the puncture site.

13. The multi-functional catheter system according to claim 3, wherein a taper of the mid-section (136) covers a range of diameters that are color-indicated on a surface of the adapter.

14. The multi-functional catheter system according to claim 1 wherein the adapter comprises a spherical protrusion (138) positioned below and around a proximal end (132) of the adapter that during insertion of the adapter into a puncture site (402) exerts a sealing force on an opening of the puncture site (402), thereby enabling hemostatic sealing, and further wherein the spherical protrusion is provided with color-indications that are color-coded to correspond to French size units used to indicate sizes in medical procedures.

15. The multi-functional catheter system according to claim 1 wherein the adapter further comprises a lockable cuff (139) coaxially received in an upper position on the adapter (130), and configured for placement in a lower position on the adapter that is located in a puncture site (402) of a patient to be treated, thereby exerting a sealing force on an opening of the puncture site that enables hemostatic sealing.

16. The multi-functional catheter system according to claim 1, wherein the adapter (130) is formed from thermoplastic elastomers, and wherein the thermoplastic elastomers of the adapter (130) include at least one or more of acrylonitrile butadiene styrene, polytetrafluoroethylene, fluorinated ethylene propylene, polyvinyl chloride, polyamide, polyurethane, polyethylene, low density polypropylene, high density polypropylene, polyethylene imine, and silicone.

17. The multi-functional catheter system according to claim 1, wherein the adapter (130) is formed from a combination of malleable and elastic elastomers.

18. The multi-functional catheter system according to claim 1, wherein the distal end (131), mid-section (136, 137) and proximal end (132) of the adapter (130) are formed from same or different thermoplastic elastomers.

19. (canceled)

20. The multi-functional catheter system according to claim 1, wherein coupling of the adapter (130) to the distal shaft (120) extends a first usable length (333) to a second usable length (334), by an amount determined through a length (300) of the adapter.

21. The multi-functional catheter system according to claim 1, wherein the guidewire lumen extends from the guidewire port (152) at the hub (150) through a single-lumen portion (141) of the proximal catheter extension shaft and a single lumen portion (124) of the distal catheter shaft to the catheter tip (110).

22. The multi-functional catheter system according to claim 1, wherein the proximal and distal catheter shafts (140, 120) are joined together at a sealing fitting (143) that enables the proximal catheter extension shaft (140) to slide into the distal catheter shaft (120) while enabling fluid impermeability of the guide wire lumen (141, 124).

23. The multi-functional catheter system according to claim 1, wherein the proximal catheter extension shaft (140) and distal catheter shaft (120) include mechanical position-maintaining features (121, 122, 123) that maintain the proximal catheter extension shaft at a constant length prior to and following usable length adjustment.

24. The multi-functional catheter system according to claim 23, wherein the position-maintaining features comprise at least one of:
a first set of positioning markers (125) positioned at regular intervals along the outer surface of the distal catheter shaft (120), each marker embedded flush within the shaft surface; the inner shaft surface below the marker protruding onto the sealing fitting (143) in order to impede sliding of the proximal catheter extension shaft (140) relative to the distal catheter shaft (120);
a second set of positioning markers (125, 126, 127) positioned at regular intervals along the outer surface of the proximal catheter extension shaft (140),
a set of annular striations (221, 222) embossed into an outer surface of the shaft, wherein the striations extend to an inner surface of the guidewire lumen (124) of the distal catheter shaft (120) that in turn, are complementary in shape and position to the positioning markers (125, 126, 127), into one or more of which one or more of the positioning marker fits in order to impede sliding of the proximal catheter extension shaft (140) relative to the distal catheter shaft (120), and further
wherein the position-maintaining features are formed from a combination of elastomeric, radiopaque, colored or pigmented materials and wherein the regular intervals between the position-maintaining features determine an amount of usable-length-adjustment of the multi functional catheter system.

25. (canceled)

26. The multi-functional catheter system according to claim 1, wherein the distal catheter shaft 120 and proximal catheter extension shaft further includes two or more orientation maintaining features (224, 226 and/or 225, 227) that reversibly lock or unlock an orientation of the proximal catheter extension shaft 140 with respect to the distal catheter shaft 120, and in turn enable the transmission of torque or rotational force by a treatment provider to a catheter tip.

27. The multi-functional catheter system according to claim 1, wherein the catheter tip (110) is formed from a combination of elastomeric and radiopaque materials.

28. The multi-functional catheter system according to claim 27, wherein the elastomeric material of the catheter tip (110) is provided in a first shape, that is elastically or plastically deformable to a second shape by insertion or removal of a guidewire having a higher stiffness than the elastomeric material thereby enabling shapeability of the catheter tip

29. The multi-functional catheter system according to claim 27, wherein an elastic modulus of the radiopaque material of the catheter tip (110) is at least equivalent or higher than an elastic modulus of the elastomeric material.

30. The multi-functional catheter system according to claim 28, wherein the transition between the first and the second shape of the catheter tip (110) occurs prior to or during a procedure, and enables steering of the catheter tip in a vasculature of a patient.

31. The multi-functional catheter system according to claim 27, wherein an elastic modulus or hardness of the catheter tip (110) exceeds a range of elastic moduli or hardness present in the vasculature thereby enabling crossing capability of the tip.

32. The multi-functional catheter system according to claim 27, wherein an elastic modulus or hardness of the catheter tip (110) does not exceed a range of elastic moduli or hardness present in the vasculature thereby enabling atraumatic maneuvering capability of the tip.

33. The multi-functional catheter system according to claim 1, wherein the guide wire lumen (141, 124) of the proximal and distal catheter shaft extends into a single lumen (129) of the catheter tip (110).

34. The multi-functional catheter system according to claim 1, wherein the guide wire lumen (141, 124) of the proximal and distal catheter shaft extends into a dual lumen (129, 115) of the catheter tip (110), and wherein one of the two lumens (129,115) extends from the catheter tip (110) into a guidewire port (117) present on a lateral surface of the catheter shaft.

35. The multi-functional catheter system according to claim 34, wherein the dual lumen (129, 115) of the catheter tip (110) extends into a single lumen (114/116) that enables containing and mixing of physiological, therapeutic and diagnostic fluids.

36. The multi-functional catheter system according to claim 1, wherein a position of the guidewire lumen is indicated by one or more radiopaque materials (112, 113) that in turn indicate one or more resting positions of one or more guidewires (101, 102).

37. The multi-functional catheter system according to claim 34, wherein the dual lumen (129, 115) of the catheter tip (110) enables the simultaneous use of one or more guidewires (101, 102) in a rapid-exchange (RX) or over-the-wire (OTW) configuration.

38. The multi-functional catheter system according to claim 34, wherein the dual lumen (129, 115) of the catheter tip (110) enables the simultaneous positioning of one or more guidewires (101, 102) in the one or more resting positions in the catheter tip (110), thereby enabling the simultaneous or consecutive use of the guidewires without loss of access to a treatment site or requiring the exchange of the one or more guidewires.

39. The multi-functional catheter system according to claim 38, wherein the positioning of one of the one or more guidewires (101, 102) in one of the one or more resting positions in the catheter tip (110) temporarily blocks the guidewire lumen (115), thereby enabling the focal delivery of physiological, therapeutic and diagnostic fluids across the guidewire lumen (129).

40. A method for treating a vascular pathology with a multi-functional catheter system according to claim 1, the method comprising:
introducing a guide wire through a puncture site into a patient's blood vessel and positioning the guide wire at a first position;
inserting a catheter tip and distal catheter shaft portion of a multi-functional catheter system into the patient's blood vessel over the guide wire;
sliding an adapter coupled to a distal end of a hub of the multi-functional catheter system to a position that couples the adapter to a proximal end of the distal catheter shaft;
advancing the distal catheter shaft in unison with the adapter into the puncture site until the adapter is positioned within the puncture site;
maintaining the position of the adapter within the puncture site while advancing the distal catheter shaft in unison with the proximal catheter extension shaft, to position a tip of the catheter at a first site within the patient's blood vessel;
changing the position of the guide wire within the patient's blood vessel;
extending or contracting the multi-functional catheter, and
repositioning the catheter tip of the catheter at a second site within the patient's blood vessel.
